(19) United States
(12) Patent Application Publication
(10) Pub. No.: US 2023/0300584 A1
(43) Pub. Date: Sep. 21, 2023

(54) PERSONAL DEVICE, A MONITORING SYSTEM AND METHODS FOR SELF-ORGANIZING A NETWORK OF NODES BY ASSIGNING DIFFERENT ROLES TO THE NODES

(71) Applicant: Pink Nectarine Health AB, Stockholm (SE)

(72) Inventors: Tobias LINGHAMMAR, Enebyberg (SE); Martin LINREY, Sollentuna (SE)

(73) Assignee: Pink Nectarine Health AB, Stockholm (SE)

(21) Appl. No.: 18/015,305

(22) PCT Filed: Jul. 9, 2021
(86) PCT No.: PCT/SE2021/050716

§ 371 Date: Jan. 9, 2023

(30) Foreign Application Priority Data
Jul. 9, 2020 (SE) 2050878-4

(51) Int. Cl. H04W 4/80; H04L 41/12

(52) U.S. Cl. CPC H04W 4/80; H04L 41/12

(57) ABSTRACT

Methods, a monitoring system, a self-organizing network of nodes and a plurality of portable personal devices. The assignment of roles for the network nodes is associated with a specific individual personal device and is done on two levels, control level and connect level. The role assigned to a network node may be dynamically changed based on data obtained by the personal devices and/or the node network.

### TECHNICAL FIELD

The present invention relates to methods, a monitoring system, a self-organizing network of nodes and a portable personal device. Specifically, the present invention relates to methods for assigning roles to network nodes and dynamically changing the assignment of roles, a system for monitoring a plurality of portable personal devices with short-range communication capabilities for communicating with network nodes.

### BACKGROUND

Systems for monitoring such individuals, e.g. elderly, sick or injured people, and automatically issuing alarms have been used for some time. Monitoring systems for monitoring such individuals often comprise a personal device (e.g. a wristband) comprising sensors, the signals of which are analysed to determine whether help is needed, e.g. due to a fall.

US6433690 discloses a method and system for recording acceleration and body position data from elderly or disabled persons. The fall monitoring system includes signal feature extraction and interpretive methods for characterizing accelerations and body positions during fall events. The system can detect health and life-threatening fall events in elderly persons and can autonomously notify nursing personnel or family members that the person is in need of immediate assistance. The monitoring of a person’s fall is performed by using an accelerometer in a monitoring device carried on the person, which monitoring device samples the person’s body angle and body acceleration.

US2019/0215244 describes a Self-Organizing Network (SON) for Internet of Things (IoT), where an IoT device may be selected to be an “loT coordinator”. A coordinator may e.g. perform the roles of managing a neighboring IoT device and forwarding a packet. This SON method includes receiving neighboring device information from at least one neighboring loT device of an IoT device, updating device information of the loT device based on the received neighboring device information, and determining whether the loT device is a coordinator candidate based on at least one of a number of network interfaces, a number of neighbor links connected to the network interfaces, or a ratio of remaining energy, which is included in the device information.

The managing of the loT devices in US2019/0215244 is performed by neighboring loT devices and, thus, relies on the presence of other loT devices within short-range communication range and is also sharing short-range wireless transmission medium and protocol with other loT devices thus is dependent on the currently available short-range bandwidth of those neighboring loT devices, e.g. the current number of other links connected to the network interface of those neighboring loT devices.

There is a need for a more scalable and load-balanced monitoring system and network which enables both faster responses to events and sensor data and provides a higher bandwidth capacity for the communication between the personal devices and the units making the decisions on behalf of the personal devices, e.g. the control units used for collecting data for monitoring the individuals.

More specifically, there is a need for a more scalable monitoring system comprising a self-organizing network which does not rely on the presence of other loT devices within short-range communication range for making decisions on behalf of other loT devices and which is less dependent on sharing short-range wireless transmission medium, interface and protocol with other loT devices. As an example, there is a need for a monitoring system and network which is less dependent on the currently available short-range bandwidth of those neighboring loT devices, e.g. the current number of other links connected to the network interface of those neighboring IoT devices.

### PROBLEMS WITH THE PRIOR ART

It is difficult to design a system for monitoring a group of personal devices carried by individuals moving between different locations or rooms of a facility which is highly scalable and provides reliable and fast responses to obtained sensor data, event data and inputs from the monitored individuals, yet is robust in that each of the personal devices always has a reliable short-range wireless connection to the node network which compete less for bandwidth.

There is thus a need for an improved system and method for monitoring a plurality of individuals within a facility.

### SUMMARY

The above described problem is addressed by the claimed system for monitoring individuals within predetermined facilities by assigning different roles to the nodes of the network of the monitoring system.

The technology disclosed relates to methods, a monitoring system comprising a self-organizing network of nodes and a plurality of personal devices. According to aspects, the assignment of roles for the network nodes is associated with a specific individual personal device and is done on two levels, a control level through the assignment of a controller node and a connect level through the assignment of a connection node. In embodiments, the role assigned to a network node may be dynamically changed based on data obtained by the personal devices and/or the node network.

The monitoring system comprising the node network of the technology disclosed is highly scalable and provides reliable and fast responses to obtained sensor data, event data and inputs from the monitored individuals, yet is robust in that each of the personal devices always has a reliable short-range wireless connection to the node network which is not disturbed or compete for short-range wireless bandwidth with other devices connected to the same node of the node network.

The monitoring system and methods of the technology disclosed is a scalable solution in providing dynamic reconfiguration of the node network during its operation, yet is robust and reliable in that each of the personal devices always is provided a reliable short-range wireless connection to the network. This is achieved by assigning a controller node to each personal device which is responsible for assigning a connection node to the personal device and dynamically changing connection node for the personal device, where the connection node is responsible for establishing a short-range wireless connection with the personal device.

According to aspects of the technology disclosed, the other network nodes shares or forwards any received or obtained data or information, e.g. status data, sensor data, event data or input data, which is received from or associated with a certain personal device to the controller node for the personal device. This allows for redundancy and, thus, safer and more accurate decision-making by the controller node, as well as provides faster responses to events and obtained sensor data in that all data or information associated with a certain personal device is quickly available to the one controller node having the sole responsibility for taking actions and making decisions on behalf of the personal device, e.g. fast responses in terms of making a decision to set an alert, change a health state and/or change connection node for the personal devices.

According to aspects of the technology disclosed, there is only one controller node for each personal device and the system is configured so that each of the other network nodes shares or forwards any received or obtained data or information, e.g. status data, sensor data, event data or input data, associated with a certain personal device to the controller node for the personal device. This provides for redundancy and, thus, safer and more accurate decision-making by the controller node as well as faster responses to events and obtained data in that all data or information associated with a certain personal device is received by the one controller node having the sole responsibility for making decisions and taking actions on behalf of the personal device. The fact that the controller node assigned to a personal device according the technology disclosed has the sole responsibility for making decisions and taking actions on behalf of the personal device and that the other nodes continuously shares or forwards obtained data or information associated with the personal device to the controller node assigned to the personal device enables a decision process which is very fast.

In aspects, the technology disclosed proposes a monitoring system and node network for dynamically assigning roles to the nodes of the network on two levels, a control level and connect level. Each of the personal device may then be assigned only one network node, on the control level, to be controller node for the personal device and only one network node, on the connect level, to be the connection node for the personal device. The assigned controller is then responsible for dynamically assigning only one connection node for the personal device at a time. The controller node assigned to a personal device may be assigned according to a common decision model known to and used by the at least three network nodes and following the sharing or distribution of data associated with the personal device. In embodiments, the data shared between the nodes may then be used as input values to the common decision model for dynamically changing controller node for the personal device. The two level self-organizing network according the technology disclosed, where a controller node assigned to a personal device has the sole responsibility for making decisions and taking actions on behalf of the personal device and the connection nodes assigned to the personal device is responsible for establishing a short-range wireless connection with the personal device and then is configured to continuously share or forward data obtained through the established connection with the personal device to the controller node, enables a decision process which is very fast, yet is robust in that the connection node is dynamically assigned to the personal device. As examples, the fast decision process enabled by the two level self-organizing network and monitoring system according the technology disclosed results in fast routing of data in the node network and a fast handover process when dynamically assigning a new connection node to a personal device based on obtained data. The node network according to the technology disclosed is typically a fast and highly scalable IP-based network with IP-based communication links connecting the nodes which are logically and/or physically separated from the short-range wireless communication links, e.g. Bluetooth links, established between the connection assigned to a personal device and the personal device. Thus, the fast and scalable IP-based network connecting the network nodes is typically using a different communication protocol, e.g. an IP-based communication protocol, from the communication protocol, e.g. a Bluetooth protocol such as BLE, used for the short-range wireless communication links between the connection nodes and the respective personal device.

According to aspects of the technology disclosed, there is only one connection node for establishing a short-range wireless connection with the personal device and the system is configured so that the controller node for the personal device has the sole responsibility for changing connection node for the personal devices. This provides for a fast response yet robust and scalable solution for monitoring personal devices, e.g. wristbands, necklaces or clips carried or worn by individuals moving between locations or rooms of a facility such as patients in a nursing home. The established short-range wireless connection is then the only connection between the network and the personal device. According to this embodiment, the personal device may then still transmit short-range wireless broadcast signals to provide the other network nodes within short-range wireless communication range with status data, obtained sensor data, event data and/or input data associated with the personal device.

In certain embodiments of the technology disclosed, the monitoring system is configured so that the connection node assigned to a personal device is never the assigned controller node for the personal device.

In embodiments of the technology disclosed, the monitoring system is configured so that the connection node assigned to a personal device is never the currently assigned controller node for the personal device, yet the controller node and connection node assigned to a personal device may be dynamically changed so that a node that previously was connection node for a personal device may be the controller node for the personal device, and vice versa.

In certain aspects and embodiments of the technology disclosed, the monitoring system is configured so that the connection node assigned to a personal device may, from time to time, also be the currently assigned controller node for the personal device. According to these embodiments, the connection node assigned to the personal device may be dynamically changed so that a network node that previously was both controller node and connection node for the same personal device may later on be only controller node for the personal device and not the controller node for the personal device.

In certain aspects and embodiments of the technology disclosed, the monitoring system is configured so that the connection node assigned to a personal device may, from time to time, also be the currently assigned controller node for the personal device. According to these embodiments, both the connection node and controller node assigned to the personal device may be dynamically changed so that a network node that previously was both controller node and connection node for the same personal device may later on be neither connection node nor controller node for the personal device.

According to aspects of the technology disclosed, the wireless transmissions of data between the personal devices and the nodes of the network of nodes are thereby separated from the transmissions, e.g. wireless transmission, of data between the nodes of the node network. The data shared between the nodes of the network may then be transmitted over an IP-based network, e.g. WiFi or Ethernet, and the data transmitted from the personal devices to the node network and the data exchanged between the personal devices and the individual nodes of the network may be transmitted via short-range wireless communication, e.g. using a short-range wireless communication protocol such as Bluetooth or other protocols providing similar short-range communication range.

According to aspects of the technology disclosed, the data transmitted from the personal devices to the node network and between the personal devices and the network nodes may then contain sensor data or event data obtained and transmitted by the personal devices and then received by at least one network node within short-range wireless communication range. The transmission of data between the at last three network nodes may include at least one of the transmission of data associated with a certain personal device which is transmitted or forwarded from one of the nodes to the assigned controller node for the personal device, transmission of data which is shared between the nodes prior to assigning a node to be the controller node for a personal device and the transmission of data from the controller node to inform and/or instruct a (new) connection node which is assigned, e.g. dynamically assigned, by the controller node to be the connection node for a personal device and which is assigned to establish a short-range wireless connection with the personal device.

The technology disclosed provides a more scalable monitoring system comprising a node network that does not rely on the presence of other personal devices within short-range communication range for making decisions on behalf of a personal device and, since the personal devices connected to the node network are not sharing their short-range wireless communication interface to the node network with short-range wireless connections to other personal devices, is less dependent on the currently available short-range wireless bandwidth. The technology disclosed comprising a node network where the nodes are transmitting obtained data associated with a certain personal device to the controller node responsible for the personal is therefore less dependent on the current number of other short-range wireless connections to the communication interface of the personal devices.

According to aspects, the controller node having the sole responsibility for a certain personal device is responsible for assigning and dynamically changing which of the at least three network nodes is the connection node for the personal device. The controller node may then decide to change connection node for a personal device the controller node is responsible for at least partly based on data received from the other nodes, e.g. measured signal strengths of short-range broadcast signals or messages transmitted by the personal device and which are received by any of the other nodes which are within short-range communication range with the personal device. The controller node may thereby see to that the personal device always has the best possible or available short-range wireless connection, e.g. Bluetooth connection, to the network of nodes which in turn may be communicatively coupled to a backend system. This provides for a scalable monitoring system which responds quickly to events, obtained sensor data and the movements of the individuals in that data is shared between the nodes, yet is robust in that each of the personal devices is continuously and always provided with a reliable short-range wireless connection to the network.

The dynamic assignment of connection node by the controller node by taking into account at least one of signal strengths of broadcast signals measured by the other nodes and the number of current short-range connections to the other network nodes also provides improved load-balancing in the network. The controller node, or a backend system communicatively coupled to the controller node, may further use a more advanced load balancing algorithm to further improve the load-balancing in the network where the load-balancing algorithm may consider at least one of the number of personal devices each of the network nodes is controller node for, the number of short-range wireless connections in a certain location or geographical sub-area of the facility and the number of personal devices in a certain location or geographical sub-area of the facility.

In aspects, the technology disclosed relates to a monitoring system comprising a node network which is self-organizing by assigning a controller node to each personal device within short-range communication range with at least one of the nodes of the network.

In embodiments, the assignment of a controller node may use a common decision model which is known to the nodes of the node network where the decision model may, or may not, use input or parameter data values obtained from the personal devices and/or other nodes of the network to determine which of the at least three nodes is going to be the controller node. The input or parameter data values may change the output from the use of the common decision model and the determining of which of the network nodes is going to be the controller node for a personal device. In some embodiments, the controller node assigned to a certain personal device may be dynamically changed at least partly based on obtained input or parameter data values.

In embodiments, the assignment, by the controller node, of a connection node may be at least partly based on signal strengths of short-range wireless broadcast signals which are measured by a plurality of network nodes within short-range communication range with the personal device transmitting the short-range wireless broadcast signals. The assignment of connection node by the controller node may further be based on or use a load-balancing algorithm which may consider at least one of the number of personal devices each of the network nodes is controller node for, the number of short-range wireless connections in a certain location or geographical sub-area of the facility and the number of personal devices in a certain location or geographical sub-area of the facility.

In embodiments, the technology disclosed relates to a personal device, a system comprising at least three network nodes and methods in a system comprising a Self-Organizing Network (SON) and a plurality of personal device, e.g. a plurality of wristbands worn by individuals such as patients.

In aspects, the technology disclosed relates to methods for dynamically assigning roles to network nodes, a system for monitoring a plurality of personal devices and a portable personal device with short-range communication capability for communicating with network nodes.

The monitoring system may comprise a memory configured to store obtained or received data associated with the individual personal devices, e.g. data detected or registered by a sensor of the individual personal device or measurement data provided by network nodes of the monitoring system. The monitoring system may further comprise a data processing unit and computer program code control logics.

In aspects, the technology disclosed relates to a monitoring system including a network comprising at least three network nodes configured to transmit data to the other nodes via the network and a plurality of personal devices with short-range wireless communication capabilities for communicating with the at least three network nodes.

The plurality of personal devices are each assigned only one controller node among the at least three network nodes, and the respective controller node is responsible for assigning a connection node among the at least three network nodes to establish a short-range wireless communication connection with a personal device the controller node is responsible for.

In embodiments, the monitoring system is configured so that each of said plurality of personal devices is assigned only one controller node which has the sole responsibility for the personal device.

In embodiments, said monitoring system is configured so that each or at least one of the at least three network nodes may be the assigned sole controller node for a plurality of personal devices.

In embodiments, said monitoring system including the at least three network nodes is configured to use a common decision model which is known to each of the at least three nodes for determining which of the at least three nodes is going to be the controller node assigned to the respective personal device.

In embodiments, said common decision model use input values or parameters which are continuously and dynamically changed at least partly based on data distributed and shared between the network nodes, thereby allowing for the determining of which of the nodes among the at least three nodes is going to be the controller node for the personal device to be dynamically changed at least partly on data distributed and shared between the network nodes. The network for distributing and sharing data between the network nodes is typically an IP-based network providing for improved scalability, e.g. a WiFi or Ethernet.

In embodiments, said monitoring system is configured so that each assigned network node to be connection node for a personal device only has one established short-range wireless communication connection with the node network at a time.

In embodiments, said plurality of personal devices are each configured to be transmitting short-range wireless broadcast signals while having an established short-range wireless communication connection with their respective connection node.

In embodiments, the only one controller node assigned to the respective personal device is configured to assign and dynamically change which of the at least three network nodes is currently the only one connection node for the personal device at least partly based on at least one of contents and received signal strengths of at least one short-range wireless broadcast signal transmitted from the personal device and which is received by a plurality of other nodes than the controller node.

In embodiments, the only one controller node assigned to the respective personal device is configured to receive data from a plurality of other nodes than the controller node via an IP-based network connecting the at least three network nodes, and wherein said data received via said IP-based network include data indicating the measured signal strength values of short-range wireless broadcast signals transmitted by the respective personal device and which are received and measured by the plurality of other nodes.

In embodiments, the only one controller node assigned to the respective personal device is further configured to change the only one connection node for the personal devices the only one controller node is responsible for at least partly based on said data indicating the measured signal strength values of short-range wireless broadcast signals transmitted by the respective personal device and which are received and measured by the plurality of other nodes.

In embodiments, the network comprises at least three network nodes configured to transmit data to the other nodes via the network is an IP-based network separated from and using a different communication protocol than used for the short-range wireless connections established between the personal devices and the network nodes. The short-range wireless capabilities of the plurality of personal devices for communicating with the at least three network nodes may then typically be Bluetooth communication capabilities, or similar short-range wireless capabilities.

In embodiments, said plurality of personal devices may include a plurality of wristbands, necklaces or clips, each may be provided with a sensor or an input means and worn by an individual such a patient of a nursing home.

In aspects, the technology disclosed relates to a method in a monitoring system including a network comprising at least three network nodes and a plurality of personal devices with short-range wireless communication capabilities, said method comprising:
a. assigning only one node among the at least three network nodes to be the controller node having the sole responsibility for a particular personal device;
b. assigning, by said controller node, a connection node for establishing a short-range wireless communication connection between said connection node and said personal device;
c. establishing, by said assigned connection node, a short-range wireless communication connection with said personal device;
d. transmitting, from said personal device and via said established short-range wireless communication connection, a message to said connection node;
e. receiving, by said connection node, said message, wherein said message contains data including at least one of status data, sensor data and event data associated with said personal device;
f. transmitting, from the assigned connection node to the sole controller node for said personal device, said received message or a message including data corresponding to, or at least partly based on, said at least one of status data, sensor data and event data associated with said personal device; and
g. transmitting, from said controller node to at least one of at least one of the other at least three network nodes and a backend system, a message containing data adapted to be used for at least one of controlling and monitoring the personal device, wherein said message is at least partly based on said data corresponding to, or at least partly based on, said at least one of status data, sensor data and event data associated with said personal device.

In embodiments, the short-range wireless communication connection is the only currently established communication connection between the network nodes and said personal device.

In embodiments, each of the plurality of personal devices is assigned only one of the at least three network nodes to be the controller node for the respective personal device.

In embodiments, each of the plurality of personal devices is assigned, by their respective controller node, only one of the at least three network nodes to be the connection node for the personal device.

In embodiments, each of the at least three network nodes has no more than one established short-range wireless connection with a personal device at a time.

In embodiments, the above method further comprises distributing or sharing data between the at least three network nodes via an IP-based network not using the same short-range wireless communication protocols used by the personal devices for communicating with the node network. The distributing or sharing of data between the nodes is preceding the assignment of the controller node, and the data distributed or shared between the at least three network nodes is further used for determining which of the at least three nodes is going to be the controller node for the personal device.

In embodiments, the distributing or sharing of data between the nodes is preceding the assignment of the controller node, and the data distributed or shared between the at least three network nodes is used for determining which of the at least three nodes is going to be the controller node for the personal device.

In embodiments, the method further comprises determining which of the at least three nodes is going to be the controller node for the personal device by using a common decision model which is known to each of the at least three nodes.

In embodiments, the common decision model use input values or parameters which are continuously and dynamically changed at least partly based on data distributed and shared between the network nodes, thereby allowing for the determining of which of the nodes among the at least three nodes is going to be the controller node for the personal device to be dynamically changed at least partly on data distributed and shared between the network nodes.

In embodiments, the method further comprises:
a. transmitting, from said personal device and via said established short-range wireless communication connection, a message to said assigned connection node;
b. receiving, by said connection node, said message, wherein said message contains data including at least one of status data, sensor data and event data associated with said personal device;
c. transmitting, from the assigned connection node to the sole controller node for said personal device, said received message or a message including data corresponding to, or at least partly based on, said at least one of status data, sensor data and event data associated with said personal device; and
d. transmitting, from said controller node to at least one of at least one of the other at least three network nodes and a backend system, a message containing data adapted to be used for at least one of controlling and monitoring the personal device, wherein said message is at least partly based on said data corresponding to, or at least partly based on, said at least one of status data, sensor data and event data associated with said personal device.

In embodiments, the method further comprises the following step prior to transmitting said message containing data adapted to be used for at least one of controlling and monitoring the personal device:

e. determining, by said controller node, whether to make any decisions or take any actions associated with and/or on behalf of said personal device at least partly in response to said received message and at least partly based on said received at least one of status data, sensor data and event data associated with said personal device.

In embodiments, the method further comprises:

f. transmitting, from said personal device, short-range wireless broadcast signals.

In embodiments, said short-range wireless broadcast signals are transmitted while said personal device has an established short-range wireless communication connection with the currently assigned connection node.

In embodiments, the method further comprises forwarding, by at least a plurality of nodes within short-range wireless communication range with the personal device, at least a portion of the contents of said short-range wireless broadcast signals to the other network nodes. The at least a portion of the contents of said broadcast signals may then be used by the network of nodes as input values to the for determining which of the at least three nodes is going to be the controller node for the personal device.

In embodiments, the method further comprises forwarding, by at least a plurality nodes within short-range wireless communication range with the personal device, at least a portion of the contents of said short-range wireless broadcast signals to the controller node. The contents of said broadcast signals may then be adapted to be used by the controller node for determining which of the at least three nodes is to be assigned connection node for the personal device.

In various embodiments, the method further comprises:
a. receiving, by a plurality of nodes within short-range wireless communication range with the personal device, said short-range wireless broadcast signals;
b. measuring, by each of said plurality of nodes, signal strengths of said received short-range wireless broadcast signals;
c. transmitting, from each of said plurality of nodes and via the IP-based network connecting the at least three nodes, data to said controller node, wherein said data indicates said measured signal strengths values;
d. receiving, by said controller node and via said IP-based network, said transmitted data indicating said measured signal strengths values; and
e. determining, by said only one controller node, to change connection node assigned to said personal device at least partly based on said received data indicating said measured broadcast signal strengths values, wherein said new connection node for said personal device is a different node from the current connection node and the current controller node for said personal device.

In embodiments, the method further comprises:
f. determining, by said only one controller node, to change the only one connection node for said personal device at least partly based on said received data indicating said measured broadcast signal strengths values, wherein said new only one connection node is assigned for establishing a new short-range wireless communication connection between said new only one connection node and said personal device; and
g. establishing, by said new only one connection node for said personal device, a new short-range wireless communication connection, wherein said new short-range wireless connection is the only currently established short-range wireless connection between said node network and said personal device.

The sensor may be an accelerometer adapted to detect the movements of the portable personal device, e.g. the acceleration and movement pattern of the portable personal device.

In certain embodiments, the technology disclosed relates to methods and a system for monitoring an individual within predetermined facilities by identifying an event associated with a personal device carried or worn by the individual, e.g. with the purpose of determining whether there is a need for setting an alert. The system may comprise at least three network nodes and a plurality of personal devices each carried or worn by the respective monitored individual, where each of the plurality of personal devices comprises at least one sensor and has short-range communication capability, e.g. Bluetooth communication capability, with which the respective personal device is arranged to communicate with the network nodes. The personal device may also comprise a storage means and at least one processing device.

In embodiments, the personal device may be a wristband and the at least one sensor of the personal device may comprise at least one medical sensor, or health sensor, adapted for obtaining sensor data for determining the medical and/or physical condition of the wearer of the wristband, e.g. a medical sensor configured to sense a characteristic of the user and generate a sensor output signal.

In embodiments, the personal device may be a wristband and the at least one sensor of the personal device may comprise at least one health sensor adapted for obtaining sensor data for determining the medical and/or physical condition of the wearer of the wristband, e.g. a medical sensor configured to sense a characteristic of the user and generate a sensor output signal. The at least one health sensor, or medical sensor, may then include e.g. a body temperature sensor for obtaining sensor data for determining the body temperature of the wearer, or may include a blood pressure sensor, a heart rate sensor, a skin galvanometric level sensor etc.

In embodiments, the at least one processing device of the personal device may be arranged to receive sensor data from the at least one sensor in the personal device and determine whether the received sensor data indicates an alert state, e.g. a specific alert state among a plurality of predetermined alert states.

In certain embodiments, the alert states may comprise at least a FALL alert state to be used if a probable fall has been detected for the monitored individual and an OUT OF BED alert state to be used if it has been detected that the monitored individual is probably getting out of bed.

According to certain aspects of the technology disclosed, personal devices communicate with nodes in the node network using Bluetooth, e.g. Bluetooth broadcasts or via an established Bluetooth connection, whereas the communication between the network nodes takes place using the IP addresses of the nodes (e.g. via Wi-Fi). The network of nodes may then be self-organizing in that the nodes of the network continuously and/or automatically assigns roles to the individual nodes, where the assignment of a role is preceded by, and at least partly based on, data distributed and/or shared between the network nodes. The distributed and/or shared data is obtained by the other network nodes and is associated with the personal device to which the assignment of a role concerns.

According to aspects, the assignment of controller node for a specific personal device is preceded by first sharing data associated with the specific personal device with the other nodes using the IP-based network for the transmission of the data. The assigned controller node may then receive data associated with the specific personal device from the other network nodes and, at least partly based on the data received from the other nodes, assign a connection node for establishing a Bluetooth connection with the specific personal device. The assigned controller node is typically also responsible for changing connection node at least partly based on the data received from the other nodes, e.g. change connection node for the personal device at least partly based on signal strength values of Bluetooth broadcast signals transmitted by the personal device and which are measured by the other nodes.

The personal devices may broadcast their own universal unique identifiers (UUIDs) and seek to establish connections with all BLE devices within its Bluetooth communication range. The network nodes may be configured to store the UUIDs of all personal devices detected in their memories. The connection node assigned, by the controller node, to a certain personal device may then establish a Bluetooth connection with the personal device by transmitting a connection request via Bluetooth to the personal device where the connection request may include the unique identifier (UUID) of the personal device.

According to aspects, the network nodes may be self-organizing in that the assignment of the one controller node having the sole responsibility for a specific personal device is a distributed decision which is preceded by at least one of distributing and sharing of data between the network nodes using the IP-based network and by using a predetermined and common decision model with dynamic input values/parameters which are changed with the data distributed and shared between the network nodes.

According to aspects, the at least three network nodes may be self-organizing in that the decision to change controller node for a specific personal device is a distributed decision which is preceded by at least one of distributing and sharing of data between the network nodes using the IP-based network and by using a predetermined and common decision model which is known to all of the at least three network nodes. In embodiments, the assignment of controller node for a certain personal device, e.g. a newly added personal device, may be using a predetermined and common decision model which may continuously and dynamically change with input values/parameters taking into account the data distributed and shared between the network nodes.

According to embodiments, the network nodes may be self-organizing in that the decision to change controller node for a specific personal device is a distributed decision which is preceded by at least one of distributing and sharing of data between the network nodes using the IP-based network and by using a predetermined and common decision model with dynamic input values/parameters which are changed with the data distributed and shared between the network nodes.

According to aspects, the assignment of connection node responsible for establishing a Bluetooth connection with a specific personal device is preceded by the sharing of data associated with the personal device with the assigned controller node for the specific personal using the IP-based network for the transmission of the data to the assigned controller node. The assigned controller node may then also be responsible for changing connection node for the personal device it is sole responsible for at least partly based on the data received from the other nodes, e.g. the assigned controller node may change connection node for the personal device at least partly based on signal strength values of Bluetooth broadcast signals transmitted by the personal device and which are measured by the other nodes and subsequently transmitted to the assigned controller node using the IP-based network for the transmission of the measured signal strength data.

According to aspects of the technology disclosed, personal devices transmits data to the nodes in the node network using Bluetooth, e.g. via Bluetooth broadcasts and an established Bluetooth connection with one of the nodes, and the transmission of data, e.g. data associated with the individual personal device, takes place using their IP addresses, e.g. data received from a certain personal device or associated with a certain personal device may be shared between network nodes via the IP-based network connecting the network nodes.

According to aspects, the personal devices are not part of the IP-based network connecting the but are only configured to transmit data and/or communicate with the network nodes via Bluetooth, e.g. using an established Bluetooth connection with one of the nodes and transmitting Bluetooth broadcast signals which may be received (and measured) by any network node within Bluetooth communication range from the personal device.

According to aspects, the network of nodes may then be self-organizing in that the network continuously and automatically assigns roles to the nodes. The IP-based network, which is communicatively connecting the nodes in the network, may then be used for distributing data obtained/received by the individual network nodes to the other nodes of the network and the assignment of roles, e.g. the assignment of controller node and connection node for individual personal devices, may be at least partly based on the data or information shared or distributed between the nodes in the IP-based network. As an example, the decision by an assigned controller node for a certain personal device which of the nodes in the network is going to be the (new) connection node, i.e. the node that will establish a (new) Bluetooth connection with the personal device, may at least partly be based on data broadcasted via Bluetooth from the personal device (and/or transmitted from the personal device via an established Bluetooth connection with the current connection node for the personal device) and received by other nodes than the controller node in that the data, or information representing the data, is forwarded from the other nodes to the controller node for the personal device, e.g. data representing signal strength values for the Bluetooth signals measured by the other nodes or position data.

In embodiments, the technology disclosed relates to a method for a system including a network, e.g. a Self-Organizing Network (SON), comprising at least three network nodes configured to share data with the other nodes via an IP-based network, and a plurality of personal devices with Bluetooth communication capabilities, the method comprising:
a. assigning only one controller node among the at least three network nodes to have the sole responsibility for a personal device;
b. assigning, by said assigned controller node, a connection node for the same personal device, where controller node is responsible for assigning said connection node to said personal device;
c. establishing, by the assigned connection node, a Bluetooth connection between the connection node and the personal device; and
d. transmitting, from the personal device, data via the established Bluetooth connection to the connection node;

In embodiments, the technology disclosed relates to a method for a system including a network, e.g. a Self-Organizing Network (SON), comprising at least three network nodes configured to share data with the other nodes via an IP-based network, and a plurality of personal devices with Bluetooth communication capabilities, the method comprising:
a. assigning only one controller node among the at least three network nodes to have the sole responsibility for a personal device, wherein said step of assigning the controller node is preceded by the sharing of data between the at least three nodes via the IP-based network;
b. assigning, by said assigned controller node, a connection node for the same personal device, wherein said controller node is responsible for assigning said connection node to said personal device;
c. establishing, by the assigned connection node, a Bluetooth connection between the connection node and the personal device; and
d. transmitting, from the personal device, data via the established Bluetooth connection to the connection node;

In embodiments, the technology disclosed relates to a method for a system including a network, e.g. a Self-Organizing Network (SON), comprising at least three network nodes configured to share data with the other nodes via an IP-based network, and a plurality of personal devices with Bluetooth communication capabilities, the method comprising:
a. assigning only one controller node among the at least three network nodes to have the sole responsibility for a personal device, wherein said only one controller node for said personal device is assigned by at least one of sharing data between the at least three network node and using a predetermined and common decision model with dynamic input values/parameters;
b. receiving, by a plurality of the at least three network nodes, Bluetooth broadcast data from said personal device;
c. forwarding, by said plurality of the at least three network nodes via the IP-based network and to said assigned only one controller node for said personal device, at least one of said received Bluetooth broadcast data, data or information corresponding to said received Bluetooth broadcast data and measured signal strength values for said received Bluetooth broadcast data;
d. assigning, by said assigned only one controller node, a connection node for said personal device, wherein said controller node is sole responsible for assigning connection node to said personal device, and wherein said assigning of connection node by said controller node is at least partly based on said at least one of said received Bluetooth broadcast data, data or information corresponding to said received Bluetooth broadcast data and measured signal strength values for said received Bluetooth broadcast data;
e. establishing, by said assigned only one connection node, a Bluetooth connection between the connection node and said personal device; and
f. transmitting, from said personal device, data via the established Bluetooth connection to the connection node;

In embodiments, the above method includes that the at least three network nodes (may be e.g. at least 5, 10, 20 or more network nodes) share data or information between each other via the IP-based network, e.g. an Ethernet or Wireless Fidelity (WiFi) network. The advantages of having the network nodes sharing data via an IP-based network whereas the personal devices connect to the network via Bluetooth connections include that the data may be distributed between

In embodiments, the assignment of controller node for a personal device may then be performed by distributing data or information received from or associated with the personal device to all of the at least three network nodes prior to deciding which of the at least three network nodes is going to be the controller node for the personal device. According to embodiments, the (distributed) decision between the at least three network nodes as to which of the at least three network nodes will be the controller node, e.g. new controller node, for the personal device is made according to a predetermined scoring system which is known to all of the at least three network nodes, where the scoring system may use dynamic variable input values. As an example, the at least three network nodes, e.g. constituting a Self-Organizing Network, may use a predetermined decision model, e.g. distributed consensus model. In various embodiments, the predetermined decision model may define a static hierarchy for the nodes or may have dynamic input values/parameters, where the input values/parameters may include, for example, data related to load balancing between different parts of the network and an assessment of the current traffic load distribution between the at least three nodes of the IP-based network, obtained (current) position data and/or signal strength values for personal devices broadcasting and/or having an established Bluetooth connection to one of the at least three network nodes, the number of personal devices each of the at least three network nodes is currently controller node for and the number of personal devices each of the at least three network nodes is currently connection node for.

In embodiments, the above method includes that the plurality of personal devices (may be e.g. at least 5, 10, 20, 50 or more personal devices) all have Bluetooth communication capabilities and all transmit Bluetooth broadcast messages/signals which may be received by any of the at least three network nodes (may be e.g. at least 5, 10, 20 or more network nodes) within Bluetooth communication range.

In embodiments, the above method includes that said Bluetooth connection is the only currently established Bluetooth connection from the network to said personal device and the connection node is the only network node having an established connection link with the personal device.

In embodiments, the assignment of controller node for the personal device means that all decision associated with the personal device, e.g. the assignment of connection node and the decision to change connection node for the personal device, is made by the assigned controller node.

In embodiments, the assignment of a controller node for the personal device means that the controller node has the sole responsibility for the personal device in that the other network nodes automatically forwards, via the IP-based network connecting the network nodes, any received data or information associated with the personal device, e.g. data or information received from the personal device via an established Bluetooth connection with the connection node and/or received from any of the personal devices via Bluetooth broadcasts sent out by the personal device. In embodiments, the other nodes of the network, which are not the controller node for the personal device, may then determine that a Bluetooth broadcast was sent out by the personal device by identifying a unique token or unique identification data of the personal device. The unique identification data may then be part of the Bluetooth broadcast message/signal sent out/transmitted from the personal device.

In embodiments, the technology disclosed relates to systems and methods for monitoring an individual within predetermined facilities in order to determine whether there is a need for setting an alert.

In embodiments, the technology disclosed relates to a method for a system including a network, e.g. a Self-Organizing Network (SON), comprising at least three network nodes configured to share data with the other nodes via an IP-based network and a plurality of personal devices with Bluetooth communication capabilities, the method comprising:
a. assigning only one controller node among the at least three network nodes to have the sole responsibility for a personal device among said plurality of personal devices;
b. assigning, by said assigned controller node, only one connection node for said personal device, wherein said controller node is sole responsible for assigning said only one connection node to said personal device;
c. establishing, by said assigned only one connection node, a Bluetooth connection between said only one connection node and said personal device, wherein said Bluetooth connection is the only currently established Bluetooth connection from the network to said personal device; and
d. transmitting, from said personal device, data via said established Bluetooth connection to said only one connection node;

In embodiments, the technology disclosed relates to a method for a system including a network, e.g. a Self-Organizing Network (SON), comprising at least three network nodes configured to share data with the other nodes via an IP-based network, e.g. an Ethernet or WiFi network, and a plurality of personal devices with Bluetooth communication capabilities, the method comprising:
a. assigning only one controller node among the at least three network nodes to have the sole responsibility for a personal device;
b. assigning, by the assigned controller node, a connection node for the same personal device, where the controller node is the only node responsible for assigning the connection node to the personal device;
c. establishing, by the assigned connection node, a Bluetooth connection between the connection node and the personal device;
d. transmitting, from the personal device, data via the established Bluetooth connection to the connection node; and
e. forwarding, by the connection node and via the IP-based network, the data received from the personal device to the assigned controller node for the personal device.

In embodiments, the technology disclosed relates to a method for assigning a connection node for a personal device in a Self-Organizing Network (SON) comprising at least three network nodes configured to share data with the other nodes via an IP-based network, e.g. an Ethernet or WiFi network, and a plurality of personal devices with Bluetooth communication capabilities, the method comprising:
a. assigning one of the at least three network nodes to be the controller node with sole responsibility for a personal device;
b. transmitting, by the personal device not having an established connection with any of the at least three network nodes, Bluetooth broadcast messages/signals;
c. receiving, by a plurality of the at least three network nodes within Bluetooth communication range, at least one Bluetooth broadcast message/signal from the personal device, where the at least one Bluetooth broadcast message/signal contains unique identification data for the personal device;
d. forwarding, from said plurality of nodes to the assigned controller node via the IP-based network, position data for the personal device and/or measured signal strength values for the personal device, wherein the position data and/or measured signal strength values are obtained through said plurality of nodes receiving said at least one Bluetooth broadcast message/signal;
e. determining, by the node assigned to be the controller node with sole responsibility for the personal device, to assign only one node among the at least three network nodes to be the sole connection node for the personal device, wherein the determining by the controller node of which node will be the connection node for the personal device is at least partly based said position data and/or measured signal strength values obtained through said plurality of nodes receiving said at least one Bluetooth broadcast message/signal; and
f. establishing, by the node determined by the controller node to be connection node for the personal device, a Bluetooth connection with the personal device.

In embodiments, the technology disclosed relates to a method for changing connection node for a personal device in a Self-Organizing Network (SON) comprising at least three network nodes configured to share data with the other nodes via an IP-based network, e.g. an Ethernet or WiFi network, and a plurality of personal devices with Bluetooth communication capabilities, the method comprising:
a. assigning a first node among the at least three network nodes to be the controller node with sole responsibility for a personal device;
b. assigning a second node different from the first node to be the connection node responsible for establishing a Bluetooth connection with the personal device;
c. establishing, by the second node, a Bluetooth connection with the personal device;
d. transmitting, by the personal device, Bluetooth broadcast messages/signals;
e. receiving, by a plurality of nodes other than the first controller node and the second connection node, at least one Bluetooth broadcast message/signal from the personal device, wherein the at least one Bluetooth broadcast message/signal contains unique identification data for the personal device;
f. obtaining, by each of said plurality of nodes and from the received at least one Bluetooth broadcast message/signal, at least one of position data and at least one signal strength values of the at least one Bluetooth broadcast message/signal measured by the nodes;
g. forwarding, from said plurality of nodes to the assigned controller node via the IP-based network, position data for the personal device and/or measured signal strength values for the personal device, wherein the position data and/or measured signal strength values are obtained through said plurality of nodes receiving said at least one Bluetooth broadcast message/signal;
h. determining, by the node assigned to be the controller node with sole responsibility for the personal device, to change which of the nodes among the at least three network nodes to be the sole connection node for the personal device, wherein the new node is among the plurality of nodes and said determining by the controller node is at least partly based said position data and/or measured signal strength values obtained through said plurality of nodes receiving said at least one Bluetooth broadcast message/signal; and
i. establishing, by the node determined by the controller node to be the new connection node for the personal device, a Bluetooth connection with the personal device.

In embodiments, the technology disclosed relates to a method for assigning a connection node for a personal device in a Self-Organizing Network (SON) comprising at least three network nodes configured to share data with the other nodes via an IP-based network, e.g. an Ethernet or WiFi network, and a plurality of personal devices with Bluetooth communication capabilities, the method comprising:
a. assigning a controller node among the at least three nodes to have sole responsibility for a personal device;
b. receiving, by at least one of the at least three network nodes within Bluetooth communication range, at least one Bluetooth broadcast message/signal from the personal device, where the at least one Bluetooth message/signal contains unique identification data for the personal device;
c. distributing, from the at least one of the at least three network nodes and via the IP-based network, unique identification data for the personal device and position data and/or measured signal strength values associated with the personal device;
d. determining, between the at least three network nodes, to assign only one controller node among the at least three network nodes to be the controller node and have the sole responsibility for the personal device, wherein the decision which of the at least three nodes will be the controller node for the personal device is made according to a predetermined and distributed (consensus) decision model with dynamic variable input parameters, and wherein the decision is at least partly based on at least one of data related to load balancing between different parts of the network and/or an assessment of the current traffic load distribution between the at least three nodes of the IP-based network, the number of personal devices each of the at least three network nodes is currently controller node for and the number of personal devices each of the at least three network nodes is currently connection node for.

In embodiments, the technology disclosed relates to a method for changing the connection node for establishing a Bluetooth connection with a certain personal device in a Self-Organizing Network (SON) comprising at least three network nodes configured to share data with the other nodes via an IP-based network, e.g. an Ethernet or WiFi network, and a plurality of personal devices with Bluetooth communication capabilities, the method comprising:
a. assigning only one controller node among the at least three network nodes to have the sole responsibility for a personal device;
b. assigning, by the assigned controller node, a connection node for the same personal device, where the controller node is the only node responsible for assigning the connection node to the personal device;
c. establishing, by the assigned connection node, a Bluetooth connection between the connection node and the personal device;
d. transmitting, from the personal device, data via the established Bluetooth connection to the connection node;
e. forwarding, by the connection node and via the IP-based network, the data received from the personal device (via the established Bluetooth connection) to the assigned controller node for the personal device;
f. transmitting, by said personal device, Bluetooth broadcast messages/signals. where the Bluetooth broadcast messages/signals are received by any of the at least three node which is within Bluetooth communication range; and
g. determining, by said controller node the personal device, to change connection node for the personal device, where the decision to change connection node is at least partly based on at least one of obtained position data and measured signal strength values for Bluetooth broadcast messages/signals received by other nodes than the controller node and the current connection node, and where the new connection node for the personal device may be a network node other than the controller node and the current connection node for the personal device.

In embodiments, the technology disclosed relates to a method in a Self-Organizing Network (SON) comprising at least three network nodes and at least one sensor devices, the method comprising:
a) transmitting, by a first sensor device among said plurality of sensor devices, short-range wireless broadcast signals, wherein each of said short-range wireless broadcast signals comprise a unique identification data for said first sensor device and may be received by any network node within short-range communication range with said first sensor device;
b) receiving, at a first network node among said at least three network nodes, at least one short-range wireless broadcast signal from said first sensor device;
c) distributing, by said first network node and within said at least three network nodes, the received unique identification data for said first sensor device;
d) assigning a second network node among said at least three network nodes to be the controller node with sole overall responsibility for said first sensor device, wherein said second network node is different from said first node, and wherein said assignment of said controller node is conducted in a distributed manner among said at least three network in accordance with a predetermined consensus decision model with dynamically variable parameter values that are changed from time to time;
e) assigning, (by said second network node), a third network node among said at least three network nodes to be the connection node for said first sensor device with sole responsibility for receiving data from said first sensor device over an established short-range wireless connection with said first sensor device, wherein said third network node is different from (both said first and)said second network node, and wherein said assignment of said connection node is conducted using a predetermined decision model with dynamically variable parameter values (load balancing e.g.) that are changed from time to time;
f) transmitting, from said third network node, a connection request message to said first sensor device via short-range wireless communication;
g) transmitting, from said first sensor device and in response to receiving said connection request message from said third network node, a connection confirm message to said third network node via short-range wireless communication;
h) establishing, (by said third network node and) in response to receiving said connection confirm message from said first sensor device, a short-range wireless connection between said first sensor device and said third network node, (wherein said established connection is the sole concurrent short-range wireless connection between said first sensor device and the SON network);
i) determining, by each of said one or more mobile communication devices, at least one of a distance to the sensor device by measurement by means of phase using a plurality of received short-range wireless broadcast signals and the signal strength of at least one received short-range wireless broadcast signal;
j) sending, from each of said one or more mobile communication devices, a message including data or information indicating the respective determined distance and/or signal strength of the short-range wireless broadcast signal;
k) receiving, at the communication node, said message from the respective one or more mobile communication devices; and
I) determining, by said communication node and at least partly based on the received data or information indicating the respective determined distance and/or signal strength received from the one or more mobile communication devices, which mobile communication device is closest to the sensor device.

In aspects, the technology disclosed relates to system comprising at least three network nodes and a plurality of personal devices, where each of the personal devices is assigned/having only one controller node and only one connection node at a time. The controller node for a personal device may then be assigned by sharing data between the nodes of the network and using a predetermined decision model, e.g. a distributed consensus model which is known to all of the at least three network nodes. In embodiments, the predetermined decision model may have dynamic input values/parameters, where the input values/parameters may include, for example, data related to load balancing between different parts of the network and an assessment of the current traffic load distribution between the at least three nodes of the IP-based network.

In aspects, the node assigned by the controller node to be the connection node for a certain personal device will establish a Bluetooth connection with the personal device. According to certain aspects, this Bluetooth connection established by the assigned connection node is then the only current connection to the nodes of the network that the respective personal device has. In embodiments and if there already exists a Bluetooth connection between the personal device and another node in the network, this Bluetooth connection will be terminated/disconnected before the new Bluetooth connection with the personal device is established between the new connection node for the personal device and the personal device.

According to certain aspects, the controller node is responsible for assigning one connection node at a time to the personal device it is responsible for but the controller node is also responsible for changing which of the nodes is the connection node based on obtained data, information and/or predetermined decision models. In embodiments and when a decision by the controller to change connection node for a personal device has been made, the controller node for the personal device may also be configured to send instruction data for instructing the current connection node to terminate/disconnect the current Bluetooth connection with the personal device. Each node in the network may then be controller node for a plurality of personal devices. Each node in the network may also be the only one connection node for a plurality of personal devices and may then establish several Bluetooth connections with different personal devices in parallel.

In aspects, the technology disclosed relates to a method in a monitoring system including a network comprising at least three network nodes configured to share data with the other nodes via an IP-based network and a plurality of personal devices with short-range wireless communication capabilities, the method comprising:
a. assigning only one controller node among the at least three network nodes to have the sole responsibility for a personal device;
b. assigning, by said controller node, only one connection node for said personal device, wherein said controller node is sole responsible for assigning said only one connection node to said personal device;
c. establishing, by said assigned only one connection node, a short-range wireless communication connection between said only one connection node and said personal device, wherein said short-range wireless communication connection is the only currently established connection from the network to said personal device;
d. transmitting, from said personal device, data via said established short-range wireless communication connection to said only one connection node; and
e. transmitting, from said personal device, short-range wireless broadcast signals, wherein said short-range wireless broadcast signals are transmitted while said personal device has said established Bluetooth connection with said only one connection node.

In embodiments, the method according to the technology disclosed is comprising:
a. receiving, by a plurality of nodes other than the only one controller node, said Bluetooth broadcast signals;
b. measuring, by each of said plurality of nodes, the signal strengths values of said received Bluetooth broadcast signals;
c. transmitting, from each of said plurality of nodes and in response to receiving said Bluetooth broadcast signals, data to said only one controller node via said IP-based network, wherein said data is adapted to indicate said measured signal strengths values of said Bluetooth broadcast signals;
d. receiving, by said only one controller node and via said IP-based network, said transmitted data indicating said measured signal strengths values; and
e. determining, by said only one controller node, to change the only one connection node for said personal device at least partly based on said received data indicating said measured broadcast signal strengths values, wherein said new only one connection node for said personal device is either the assigned only one controller node or one or one of the plurality of nodes other than the controller node and is sole responsible for establishing a new Bluetooth connection to said personal device, wherein said new Bluetooth connection will be the only currently established Bluetooth connection from the network to said personal device.

In embodiments, the method according to the technology disclosed is comprising:

. determining, by said only one controller node, to change the only one connection node for said personal device at least partly based on said received data indicating said measured broadcast signal strengths values, wherein said new only one connection node for said personal device is one of the plurality of nodes other than the controller node and is sole responsible for establishing a new Bluetooth connection to said personal device, wherein said new Bluetooth connection will be the only currently established Bluetooth connection from the network to said personal device.

In embodiments, the method according to the technology disclosed is comprising:

b. determining, by said only one controller node, the current position of said personal device based on said received data indicating said Bluetooth broadcast signal strengths values measured by said plurality of nodes.

In embodiments, the method according to the technology disclosed is comprising:
c. determining, by said personal device, that a trigger event has occurred, wherein said
d. wherein each of said plurality of personal devices is configured to transmit Bluetooth broadcast signals including alarm data in response to a trigger event determined by the respective personal device to have occurred, and wherein said alarm data is adapted to indicate the occurrence of said trigger event to any node receiving the Bluetooth broadcast signal and is transmitted by the respective personal device while having an established Bluetooth connection with their respectively assigned only one connection node.

In embodiments, the plurality of personal devices is further configured to, in addition, transmit data including redundant alarm data in response to the determined occurrence of the trigger event via the established Bluetooth connection with their respectively assigned only one connection node, and wherein the respectively assigned only one connection node is configured to, in response to receiving said alarm data via said established Bluetooth connection, at least one of transmit and forward data including alarm data via said IP-based network to the respectively assigned only one controller node, where said alarm data is adapted to indicate to the only one controller node that the trigger event has occurred.

In embodiments, the system comprises at least one mobile communication device and a backend system and network configured to receive alarm data indicating the occurred trigger event from the only one controller node responsible for the personal device which determined that the trigger event occurred, and wherein said backend system and network is further configured to transmit alarm data to the at least one mobile communication device in response to receiving said alarm data from the only one controller node.

In embodiments, the backend system and network is further configured to transmit said alarm data to the at least one mobile communication device via a network separate from said IP-based network used by the at least three network nodes to exchange data.

In embodiments, the trigger event is the activation of an input means on the respective personal device.

In embodiments, the personal device is a wristband and said trigger event is the action of pressing a button on the wristband.

In aspects, the technology disclosed relates to a monitoring system including a network comprising at least three network nodes configured to transmit data to the other nodes via a network, preferably an IP-based network, and a plurality of personal devices with short-range wireless communication capabilities for communicating with the at least three network nodes. The plurality of personal devices may each be assigned only one controller node among the at least three network nodes, and the controller node is responsible for assigning only one connection node among the at least three network nodes to establish a short-range wireless communication connection with a personal device the controller node is responsible for.

In embodiments, each of the plurality of personal devices is assigned only one controller node to have the sole responsibility for the personal device, and wherein said

In embodiments, the plurality of personal devices are each configured to be transmitting short-range wireless broadcast signals while having an established short-range wireless communication connection with their respective connection node.

In embodiments, the monitoring system is configured so that each assigned network node to be connection node for a personal device only has one established short-range wireless communication connection at a time.

In embodiments, the only one controller node assigned to the respective personal device is configured to assign and dynamically change which of the at least three network nodes is currently the only one connection node for their respective personal devices at least partly based on at least one of contents and received signal characteristics of at least one Bluetooth broadcast signal transmitted from the personal device and which is received by a plurality of other nodes than the controller node.

In embodiments, the only one controller node assigned to the respective personal device is configured to receive data via the network, e.g. an IP-based network, from a plurality of other nodes than the controller node. The data received via the IP-based network may then include data indicating the measured signal strength values of Bluetooth broadcast signals transmitted by the respective personal device and which are received and measured by the respective node of the plurality of other nodes.

In embodiments, the only one controller node assigned to the respective personal device is further configured to change the only one connection node for the personal devices the only one controller node is responsible for at least partly based on said data indicating the measured signal strength values of Bluetooth broadcast signals transmitted by the personal device and which are received and measured by the plurality of other nodes.

In embodiments, the only one controller node assigned to the respective personal device is further configured to determine the current position of the personal device it is responsible for based on said data indicating the measured signal strength values of Bluetooth broadcast signals transmitted by the respective personal device and which are received and measured by the plurality of other nodes.

In embodiments, the system further comprises a backend system and network configured to receive data indicating measured Bluetooth broadcast signal strength values for a personal device from the only one controller node responsible for the personal device. The backend system and network may then be configured to determine the current position for the personal device based on the received Bluetooth broadcast signal strength values for the personal device and return the determined position to the only one controller node responsible for the personal device.

In embodiments, each of said plurality of personal devices is configured to transmit Bluetooth broadcast signals including alarm data in response to a trigger event determined by the respective personal device to have occurred, wherein the alarm data is adapted to indicate the occurrence of said trigger event to any node within short-range wireless communication range receiving the Bluetooth broadcast signal and is transmitted by the respective personal device while having an established Bluetooth connection with their respectively assigned only one connection node.

In embodiments, each of said plurality of personal devices is further configured to, in addition, transmit data including redundant alarm data in response to the determined occurrence of the trigger event via the established Bluetooth connection with their respectively assigned only one connection node. The respectively assigned only one connection node may then be configured to, in response to receiving the alarm data via said established Bluetooth connection, at least one of transmit and forward data including alarm data via said network, e.g. IP-based network, to the respectively assigned only one controller node, where the alarm data is adapted to indicate to the only one controller node that the trigger event has occurred.

In embodiments, the system comprises at least one mobile communication device and a backend system and network configured to receive alarm data indicating the occurred trigger event from the only one controller node responsible for the personal device which determined that the trigger event occurred. The backend system may then be configured to transmit alarm data to the at least one mobile communication device in response to receiving the alarm data from the only one controller node.

In embodiments, the backend system and network is further configured to transmit the alarm data to the at least one mobile communication device via a network separate from the network, e.g. IP-based network, used by the at least three network nodes to exchange data.

In embodiments, the trigger event is the activation of an input means on the respective personal device.

In embodiments, the personal device is a wristband and said trigger event is the action of pressing a button on the wristband.

### BRIEF DESCRIPTION OF DRAWINGS

Preferred embodiments of a monitoring system according to the technology disclosed will be described more in detail below with reference to the accompanying drawings wherein:

FIG. 1 schematically shows an example of facilities where a monitoring system comprising a number of network nodes have been installed, in accordance with one or more embodiments described herein.

FIG. 2 is a schematic illustration of a system for monitoring an individual within predetermined facilities.

FIG. 3 is an example flow diagram of a method for monitoring an individual within predetermined facilities, in accordance with one or more embodiments described herein.

FIG. 4 schematically illustrates a method for monitoring an individual within predetermined facilities, in accordance with one or more embodiments described herein.

FIG. 5 schematically illustrates a method for monitoring an individual within predetermined facilities, in accordance with one or more embodiments described herein.

FIG. 6 schematically illustrates a method for monitoring an individual within predetermined facilities, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

In the drawings, similar details are denoted with the same reference number throughout the different embodiments. In the various embodiments of the monitoring system according to the technology disclosed, the different subsystems are denoted. The “boxes″/subsystems shown in the drawings are by way of example only and can within the scope of the technology disclosed be arranged in any other way or combination.

The systems and ad-hoc networks known in the art, e.g. piconets or SON, does not disclose a node network where nodes share data or information which each other prior to the dynamic assignment of roles to the nodes.

The systems and ad-hoc networks known in the art, e.g. piconets or SON, does not disclose a node network where data (traffic) is separated into the communication between the nodes, which takes place using the IP address and via an IP-based network, e.g. via Wi-Fi, and the communication between the personal devices communicate and the nodes in the node network, which uses a short-range wireless communication protocol such as Bluetooth.

The systems and networks, e.g. piconets or SON, known in the art further does not disclose a monitoring system comprising a node network and personal devices with broadcast capabilities which is self-organizing and scalable by dynamically assigning roles to the individual nodes, e.g. continuously and automatically assigning roles to the nodes, where each assignment of a role to a node relates to managing and control of only one of the personal devices. In particular, the systems and network solutions known in the art are silent to dynamically assigning and changing roles to the nodes on two levels, a control level where the node has the sole responsibility for a personal device and a connection level where the node is responsible for establishing a connection or link with the personal device.

The ad-hoc networks known in the art does not disclose or describe a node network where the nodes communicate internally via their IP addresses, but where the communication between personal devices and the network nodes takes place via a short-range wireless communication such as Bluetooth. This gives a much higher bandwidth capacity for the communication between the personal devices and the network nodes. This also enables the node network to be scalable and more distributed, since communication between network nodes can take place via the IP-based network connecting the nodes.

To address the identified problems with the prior art, the technology disclosed proposes a monitoring system and node network for dynamically assigning roles to the nodes of the network on two levels, a control level and connect level.

The technology disclosed introduces a node network where the nodes communicate internally via their IP addresses, but where the communication between personal devices and nodes takes place via short-range wireless communication such as Bluetooth. This gives a better load balancing and much higher bandwidth capacity for the communication between the personal devices and the nodes. This also enables the node network to be more scalable and distributed, since communication between nodes can take place via an IP-based network.

The monitoring system comprising the node network of the technology disclosed is highly scalable and provides for safe and fast responses to events, obtained data and the movements of the individuals, yet is robust in that each of the personal devices always has a reliable short-range wireless connection to the node network which is not disturbed or compete for short-range communication bandwidth with other devices connected to the same node of the node network.

In aspects, the technology disclosed allows for faster and more accurate decision-making and improved load balancing within a network by providing methods and a monitoring system comprising personal devices and a network of nodes for dynamically assigning one controller node and one connection node for each personal device. In embodiments, the controller node for a personal device is dynamically assigned or changed based on a common decision model known to the network nodes and which may contain input values or parameters. The input values or parameters may be changed with the data distributed and shared between the network nodes. In certain embodiments of the technology disclosed, the consensus model as such with its input values or parameters may also be dynamically adjusted and changed over time.

In embodiments, the technology disclosed relates to a monitoring system comprising a node network which is self-organization by including a dynamic assignment of a node as uploader node to the internet or backend system.

In certain embodiments, the technology disclosed relates to a monitoring system comprising a node network which is self-organization by assigning a “stand-by” node to take over from the assigned node, e.g. a stand-by node which is ready to take over from an assigned controller node, an assigned connection node and/or an assigned uploader node.

In certain embodiments, the assignment of a stand-by controller node may use the same common decision model as used for the assignment of the controller node where the decision model may, or may not, use input or parameter data values obtained from the personal devices and/or nodes of the network to determine which of the at least three nodes is going to be the stand-by node. The input or parameter data values may change the determining of which of the network nodes is going to be the stand-by controller node for the personal device.

In certain embodiments, the assignment, by the controller node, of a stand-by connection node to take over from the assigned connection node may be at least partly based on signal strengths of short-range wireless broadcast signals which are measured by a plurality of network nodes within short-range communication range with the personal device transmitting the short-range wireless broadcast signals. The assignment of stand-by connection node by the controller node may further use a load-balancing algorithm which may consider at least one of the number of personal devices each of the network nodes is controller node for, the number of short-range wireless connections in a certain location or geographical sub-area of the facility and the number of personal devices in a certain location or geographical sub-area of the facility.

The faster and more accurate decision-making is at least partly achieved by the method of assigning, for each personal device, one controller node with sole responsibility for the personal device, where the controller node is provided with all data or information associated with the personal device the controller node is responsible for and which is received, obtained and/or measured by the network nodes.

The faster and more accurate decision-making may be further achieved by the controller node making all decisions for the personal device, e.g. the decision of dynamically assigning one connection node with which the personal device is establishing a short-range wireless communication link such as a Bluetooth connection. The connection node may then have an established connection with the personal device as long as the controller node considers it appropriate, e.g. as long as the signal strength measured by the connection node is above a certain threshold value and/or a signal strength measured by the connection node is higher than signal strengths measured by the other nodes of the network.

The decision by the controller node to change connection node for a personal device may be at least partly based at least one of signal strengths of broadcast signals measured by the other network nodes and the number of current short-range connections to the other network nodes. In addition to providing the personal device with a short-range wireless connection to the node network with improved signal strength values than the current connection, this also allows for improved load-balancing in the network. The controller node, or a backend system communicatively coupled to the controller node, may further use a more advanced load balancing algorithm to further improve the decision-making by the controller node and the load-balancing in the network.

In certain aspects, the technology disclosed is aimed at providing improved scalability and load-balancing over ad-hoc networks such as piconets by separating the network the nodes are using to provide each other with data from the short-range wireless transmissions the personal devices are using for providing the individual network nodes with data. According to aspects of the technology disclosed, the improved scalability and load-balancing may be further achieved in that the network nodes are using a scalable IP-based network for providing each other with data, whereas the personal device use a short-range wireless communication protocol such as Bluetooth for communicating with the network nodes.

The improved load-balancing is achieved by having the controller node with sole responsibility for a personal device dynamically assigning and changing connection node for the personal device at least partly based on load-balancing factors such as the current number of established short-range wireless connections for a node, e.g. as compared to the number of connections for other nodes, to thereby distribute the amount of short-range wireless data transmitted locally.

In certain aspects, the monitoring system and self-organizing network of the technology disclosed separating the data traffic in the IP-based network traffic from the short-range wireless communication performed by the personal devices provides an improved solution over ad-hoc networks and many state of the art self-organizing networks in that the transmission of data between the nodes used for configuring and self-organizing the network nodes, e.g. data used for assigning different roles to the nodes, is performed via the IP-based network and not via the short-range wireless protocol the personal device are using for providing the network nodes with data, thereby does not load, affect or interfere with the short-range wireless radio traffic, e.g. the Bluetooth radio traffic.

Benefits of the technology disclosed include that the system, self-organizing network and methods provide each of the personal devices with improved short-range wireless connections by continuously and dynamically changing which of the network nodes is the sole connection node having the only short-range wireless connection, e.g. Bluetooth connection, to a personal device at least partly based on measured signal strengths for the currently established short-range wireless connection and/or short-range signal strengths measured by the individual nodes of the network.

Further benefits of the technology disclosed include that the controller node is provided with all data associated with the personal device it is responsible for in that the all data or information associated with the personal device, or data or information representing or reflecting the obtained data or information, is forwarded from the other network nodes to the controller node, including the data received and measured by the currently assigned connection node for the personal device.

In embodiments, the technology disclosed provides a fast response yet robust solution for acting on an identified event by parallel redundant data transmission of event data representing or reflecting the event via both an established short-range wireless connection with one of the nodes and via short-range wireless broadcast messages from the personal device to the nodes which are within short-range wireless communication range.

Bluetooth is a short-range wireless technology standard that allows data to be exchanged between fixed and mobile devices. The technology uses short wavelength radio waves from 2.4 to 2.485 GHz. Unlike other forms of connectivity such as wi-fi or 4G, Bluetooth carries connections between devices and other devices as opposed to carrying data to and from the internet.

BLE stands for Bluetooth Low Energy and is a form of wireless communication designed especially for short-range communication. BLE is very similar to Wi-Fi in the sense that it allows devices to communicate with each other. However, BLE is meant for situations where battery life is preferred over high data transfer speeds. Wi-Fi uses multiple parts of the IEEE 802 protocol family and is designed to interwork seamlessly with its wired sibling Ethernet. Compatible devices can network through wireless access points to each other as well as to wired devices and the Internet. The different versions of Wi-Fi are specified by various IEEE 802.11 protocol standards, with the different radio technologies determining radio bands, and the maximum ranges, and speeds that may be achieved. Wi-Fi most commonly uses the 2.4 GHz (120 mm) UHF and 5 GHz

(60 mm) SHF ISM radio bands; these bands are subdivided into multiple channels. Channels can be shared between networks but only one transmitter can locally transmit on a channel at any moment in time.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via Bluetooth broadcast transmissions from the personal device to the nodes which are within Bluetooth communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via a Bluetooth broadcast message transmitted from the personal device to the nodes which are within Bluetooth communication range.

In embodiments, the technology disclosed provides a solution for dynamically and accurately determining the indoor position or location, e.g. current room, for a personal device without any use of high energy-consuming GPS in the personal devices or iBeacon technology, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine the current position or location for the personal device.

In various embodiments, the controller node may forward the obtained data and/or information representing or reflecting the measured signal strengths from each of the plurality of other nodes, and/or other movement sensor data obtained, to a backend network which determines the current position or location for the personal device.

In various embodiments, the controller may in addition use other obtained sensor data, e.g. sensor data from the movement sensor of the personal device (e.g. an accelerometer), to determine and/or calculate the current position and/or location for the personal device.

In certain embodiments, the current position or location for the personal device may at least partly be determined by a trained machine learning algorithm used by the controller node or a backend system communicatively coupled to the controller node, since machine learning is an efficient way of evaluating obtained sensor data and event data, e.g. in situations where large amounts of data is collected. The trained machine learning algorithm may then use at least the signal strength values received from the other nodes and/or movement sensor data detected by the personal device to determine the current position or location for the personal device.

In embodiments, the technology disclosed provides a roaming solution for dynamically and continuously changing the connection node for a personal device, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine to change connection node for the personal device.

In various embodiments, the controller node may forward the obtained data and/or information representing or reflecting the measured signal strengths from each of the plurality of other nodes, and/or other movement sensor data obtained, to a backend network which determines that the connection node for the personal device should be changed.

In various embodiments, the controller node may in addition use other obtained sensor data, e.g. sensor data from a movement sensor of the personal device, e.g. an accelerometer, to determine that the connection node for the personal device should be changed.

In certain embodiments, the change of connection node for the personal device may at least partly be determined by a trained machine learning algorithm used by the controller node or a backend system communicatively coupled to the controller node, since machine learning is an efficient way of evaluating sensor data in situations where large amounts of data is collected. The trained machine learning algorithm may then use at least the signal strength values received from the other nodes and/or movement sensor data detected by the personal device to determine that the connection node for the personal device should be changed.

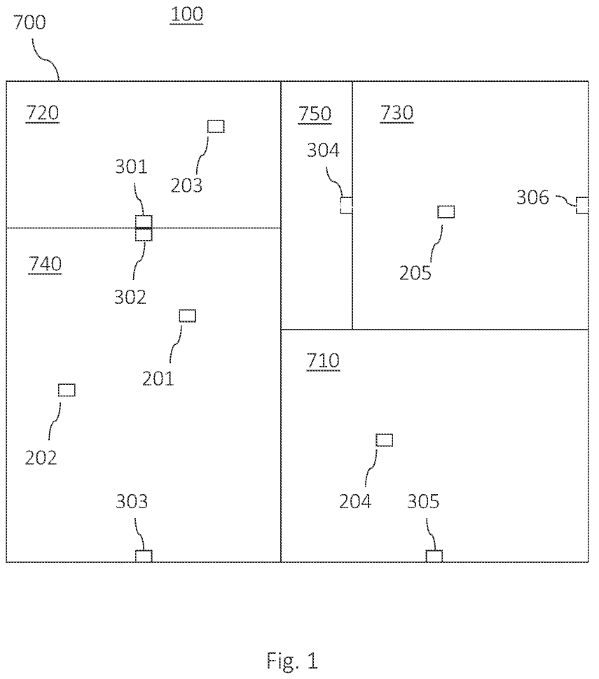

FIG. 1 schematically shows an example of a facility 700 where a monitoring system 100 comprising a number of network nodes (301, 302, 303, 304, 305, 306) has been installed, in accordance with one or more embodiments described herein. The illustrated system 100 comprises a plurality of personal devices (201, 202, 203, 204, 205) with short-range wireless communication capability. Such facility 700 may e.g. be an apartment where a monitored individual is residing. Such an apartment 700 may e.g. comprise a kitchen 710, a bathroom 720, a bedroom 730, a sitting room 740, and a hallway 750. The monitoring system 100 may in this case be used by relatives, home care companies or the like.

The facility 700 may also be other types of quarters where a monitored individual may be residing, such as e.g. a nursing home, where there may be a private space for the monitored individual, including e.g. a bedroom 730, as well as public spaces that are shared by a plurality of monitored individuals.

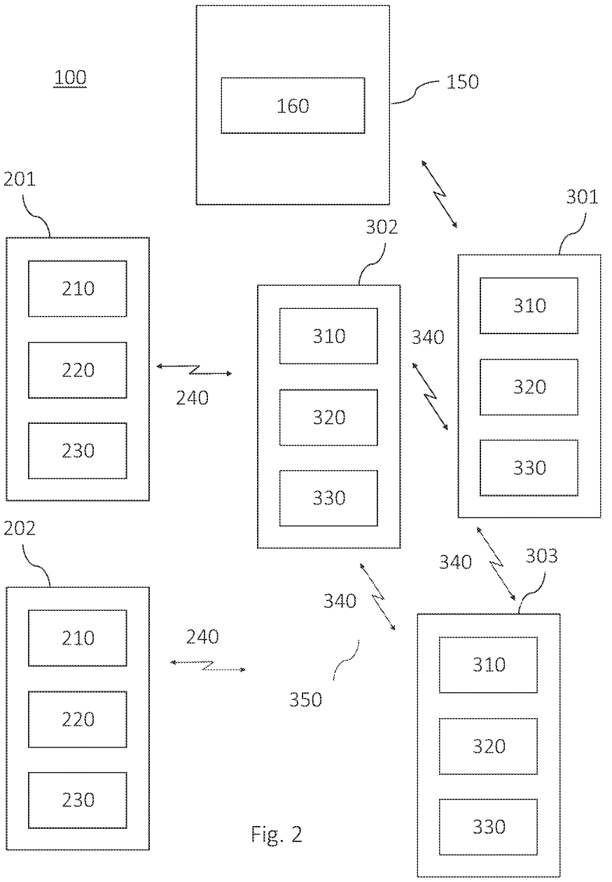

FIG. 2 is a schematic illustration of a system 100 for monitoring an individual within predetermined facilities 700, in accordance with one or more embodiments described herein. The illustrated system 100 comprises a plurality of personal devices (201, 202), a plurality of network nodes (301, 302, 303), and a remote processing arrangement 150, e.g. a backend system. The personal devices (201, 202) may be a wristband or other portable device that is carried or worn by an individual, e.g. a necklace or a clip worn by a patient in a nursing home. The illustrated personal devices (201, 202) comprise a processing device 210, at least one sensor and/or user input means 220, and a personal device communication interface 230. The at least one sensor may, for example, include a motion sensor, a temperature sensor, a health sensor such as a body temperature sensor, a pressure sensor and/or any other type of sensor and the user input means may comprise a button or voice input means that the individual can press or activate to set off an alarm and/or transmit event data indicating the occurrence of a certain event. The illustrated network nodes (301, 302, 303) comprise a node processing device 310, a storage means 320, and a node communication interface 330. The illustrated remote processing arrangement 150 comprises a remote processing device 160, which may e.g. be comprised in a web server. However, the monitoring system 100 does not necessarily comprise a remote processing arrangement 150.

Information regarding the layout of the facilities 700, such as the plan of the different rooms and the location of the node modules 300, is preferably stored in the storage means 320. The storage means 320 does not have to be comprised in the network node - it can be comprised in another part of the system such as a backend system, or be a separate device or module.

According to embodiments of the technology disclosed, the at least one processing device 310 of the network nodes (301, 302, 303) may be arranged to: receive status data, position data or signal strength values from another node of the network, or sensor data, input data or event data originating from the at least one sensor and/or user input means 220 in the personal device 200; determine whether the received data indicates a need for certain action associated with the personal device, e.g. changing connection node for the personal device or changing health state or alert state among a plurality of predetermined alert states. As an example, the alert states may comprise at least a FALL alert state, to be used if a probable fall has been detected for the monitored individual, and an OUT OF BED alert state, to be used if it has been detected that the monitored individual is probably getting out of bed.

In various embodiments, the at least one processing device 210, 310, 160 processing the sensor data, input data or event data may be the personal device processing device 210, the node processing device 310, or another processing device, such as a remote processing device 160. The at least one processing device may also be a combination of any number of processing devices, so that some of the processing takes place in one processing device and some of the processing takes place in one or more other processing devices. It is thus not necessary for all of the processing to take place in the same processing device.

If weighing factors are used, these weighing factors may be determined by a machine learning system, since this is an efficient way of evaluating complex signals in situations where large amounts of data is collected.

One way of making such a determination is to determine a probability that an alert should be set based on the received sensor data, and a probability that the determined location fulfils the predetermined location condition, and then combine these probabilities using weighing factors, which may e.g. be determined by a machine learning system.

The same applies to other combinations, such as e.g. the determination of the location by involving also signals from other sensors of the personal device such as a movement sensor 220, and/or the setting of alerts based also on activity states. These determinations may also be done by determining probabilities and using weighing factors to combine them, which may e.g. be determined by a machine learning system.

In embodiments, the change of connection node by the controller node (301) may e.g. be determined based on the received signal strength indication (RSSI) of short-range wireless broadcast signals transmitted by the personal device and which are received by the network nodes within short-range communication range with the personal device. If the RRSI of short-range wireless broadcast signals received by more than one network node is determined, also the location of the personal device may be determined based on e.g. triangulation. Machine learning may also be used for this determination. The measured RSSI values of short-range wireless broadcast signals transmitted by a personal device and/or determined location for the personal device may be used by the controller node assigned to a personal device for changing connection node for the personal device. Thus, the decision by the controller node for a particular personal device to change connection node for the personal device may be based on broadcast signal strength values measured and received from other nodes within short-range communication range with the personal device.

The personal devices 200 may also comprise other sensors 220, such as e.g. a pressure sensor, temperature sensor or medical sensor. It is known to use a pressure sensor in a personal device to detect a fall based on a change in pressure. However, a pressure sensor may also be involved in the determination of the location of the individual. If the facilities 700 comprise several floors, and the node modules 300 are arranged in the ceilings or high up on the walls, the RSSI may give erroneous results due to the signals travelling between the floors. In this situation, a pressure sensor may be used to determine the floor on which the individual is located.

In order to improve the determination of the need for e.g. changing connection node, changing health state for the individual and/or setting an alert even further, the determination of the location may be improved by involving also signals from a movement sensor 220. The movement sensor 220 may e.g. be used as a pedometer, so that the signals from the movement sensor 220 indicates how many steps the individual has taken. If the individual is determined to be in a certain location (such as e.g. the bathroom 720), and the amount of steps required for moving to this location from a previously determined location (such as e.g. the bedroom 730) is more than the amount of steps that have been detected by the movement sensor 220, it is likely that the determined location is incorrect. The at least one processing device 210, 310, 160 may thus be arranged to determine the location of the personal device 200 based also on an indication of the movement of the individual in relation to a previously determined location, based on sensor data from the at least one motion sensor 220.

The processing of the sensor data from the at least one sensor 220 may be done by a trained machine learning algorithm. When many individuals are monitored using many personal devices 200, large amounts of sensor data is collected from the sensors 220 in these personal devices 200. Very efficient evaluation of this sensor data may be done using machine learning, in order to determine a suitable machine learning algorithm.

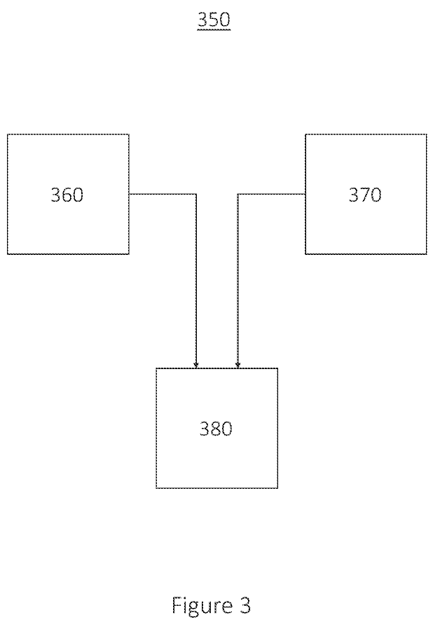

FIG. 3 is an example flow diagram of a method for monitoring an individual within predetermined facilities in order to determine whether there is a need for responding to status data, sensor data and/or event data received by the controller node, e.g. changing connection node, changing health state for the individual and/or setting an alert, in accordance with one or more embodiments described herein. The flow 350 may e.g. be as follows:

Step 360: Determine, by the controller node for a personal device 200, routing and/or load-balancing aspects or factors associated with at least two of the at least three network nodes in the network, where the routing and/or load-balancing aspects may consider at least one of the number of personal devices each of the network nodes is controller node for, the number of short-range wireless connections in a certain location or geographical sub-area of the facility and the number of personal devices in a certain location or geographical sub-area of a facility.

Step 370: Receive, by the controller node for a certain personal device, signal strength values for short-range wireless signals transmitted by a personal device 200 and which are measured and forwarded to the controller node by a plurality of network nodes 300.

Step 380: Combine these inputs into a resulting determination, by the controller node, of whether to take actions in response to the received signal strength values, e.g. to change the assigned only one connection node for the personal device 200 to the node network.

The change of assigned network node for the personal device 200 based on a combination of conditions may be done in different ways. Some of the conditions may be conditions that are set absolutely, so that if a certain condition is not fulfilled, the change of connection node for the personal device will, or will not, be determined by the controller node to occur. In other situations, a weighing of different load-balancing aspects or factors is done by the controller node or the backend system (providing the controller node with the result), e.g. the number of personal devices each of the network nodes is controller node for, the number of short-range wireless connections in a certain location or geographical sub-area of the facility and the number of personal devices in a certain location or geographical sub-area of a facility.

When the change of assigned connection node is determined based on whether the received signal strength values indicates a certain state and whether the determined location fulfils a predetermined location condition, the predetermined location condition may be an absolute condition, such that if the determined location does not fulfil the predetermined location condition, the change of assigned connection node will, or will not, be determined by the controller node to occur. However, there may instead be weighing factors, so that if the received signal strength values strongly indicates that a change of connection node for a certain personal device should occur, and/or the determined location does not strongly indicate a specific location (that does not fulfil the predetermined location condition), the connection node assigned to the personal device is changed, the health state for the individual wearing the personal device is changed and/or an alert will still be set.

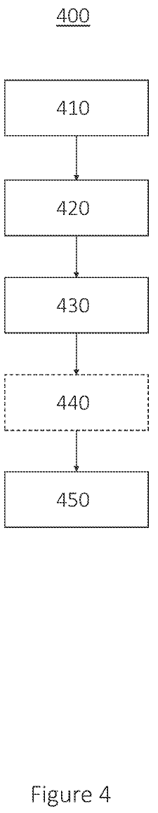

FIG. 4 schematically illustrates a method 400 for monitoring an individual within predetermined facilities 700 in order to determine whether there is a need for changing connection node for the personal device. The method 400 may include the following steps:

Step 410: transmitting short-range wireless broadcast signals from a personal device 200.

Step 420: measuring, by a plurality of network nodes 300, signal strength values of the short-range wireless broadcast signals transmitted by the personal device 200 and which are received by the plurality of network nodes 300.

Step 430: forwarding the measured signal strength values measured by the respective of the plurality of network nodes 300 to the controller node for the personal device.

Step 440: determining the location of the personal device 200 within the facilities 700 based at least on the received signal strength values together with information retrieved from a storage means 320 regarding the layout of the facilities 700 within which the monitoring of the individual takes place.

Step 450: determining to change connection node for the personal device based at least partly on the measured signal strength values of the short-range wireless broadcast signals.

In certain embodiments, the method (400) may comprise:

Step 440: determining the location of the personal device 200 within the facilities 700 based at least on the received signal strength values together with information retrieved from a storage means 320 regarding the layout of the facilities 700 within which the monitoring of the individual takes place.

In the embodiments comprising step 440, the step 450 of determining to change connection node for the personal device may be based at least partly on whether the received sensor data indicates a specific location and/or a specific node to be the new connection node and whether the determined location fulfils a predetermined location condition, indicating whether the individual is located in a certain room, such as e.g. the bedroom 630.

The combination of motion information with location information improves the determination of changing connection node for the personal device.

The determining 440 of whether the received sensor data indicates a specific location among a plurality of predetermined location may e.g. be done by a trained machine learning algorithm of the controller node or a backend system communicatively connected to the controller node, since machine learning is an efficient way of evaluating complex signals in situations where large amounts of data is collected and moving patterns of the individuals wearing the personal devices are repetitive and/or predictable.

In embodiments, the distance between the personal device 200 and the at least one network node300 may e.g. be determined based on the received or obtained signal strength indication (RSSI) of the short-range wireless signal transmitted by the personal device 200 and received by the plurality of network nodes, since the received signal strength will be lower if the distance increases. If the RRSI for more than one network node is determined, the location may be more exactly determined based on e.g. triangulation. Machine learning may also be used for this determination.

In embodiments, the determining 440 of the location of the personal device 200 is based also on an indication of the movement of the individual in relation to a previously determined location, based on sensor data from the at least one sensor 220. If the individual is determined to be in a certain location, and the movement required for moving to this location from a previously determined location is more than the movement that has been detected by the at least one movement sensor, it is likely that the determined location is incorrect.

In embodiments, the at least one sensor 220 is an accelerometer. However, other types of motion sensor may also be used.

In embodiments, the personal device 200 is a wristworn device, such as e.g. a wristband. The personal device 200 may however be any type of device that may be worn by an individual, such as e.g. a necklace or a clip.

In embodiments, communication between the personal device 200 and the at least one node 300 takes place using a personal device communication interface 230 and a node communication interface 330. In embodiments, the technology disclosed may include continuously determining and updating an activity state for the monitored individual by continuously receiving sensor data from the personal device, e.g. health sensor data obtained by a medical sensor of the personal device.

The activity state may e.g. categorize the current activity of the individual. Activity states may e.g. be LYING IN BED, LYING ON COUCH, LYING ON FLOOR, SITTING, STANDING, WALKING, RUNNING, RIDING A WHEELCHAIR, WALKING UP/DOWN STAIRS, IN THE BATHROOM. There may also be a specific activity state for when the individual is not wearing the personal device, since no other activity state can then be set. There may be a specific sensor, such as e.g. a contact sensor, a temperature sensor or a heart rate sensor, for determining whether the individual is wearing the personal device. Such a sensor may of course also be used for other purposes.

In embodiments, the technology disclosed may include continuously determining and updating a health state for the monitored individual by continuously receiving sensor data from the personal device worn by the individual, e.g. health sensor data obtained by a medical sensor of the personal device.

The determining of the activity state and/or the health state of the individual wearing the personal device may e.g. be done by a trained machine learning algorithm, since machine learning is an efficient way of evaluating complex signals in situations where large amounts of data is collected.

In embodiments, the personal device 200 may also comprise sensors, such as e.g. a motion sensor or pressure sensor. It is known to use a pressure sensor in a personal device to detect a fall based on a change in pressure. However, a pressure sensor may also be involved in the determination, by the controller, to change connection node for the personal device 200 carried by the individual. If the facilities 700 comprise several floors, and the network nodes 300 are arranged in the ceilings or high up on the walls, the RSSI may give erroneous results due to the signals travelling between the floors. In this situation, data obtained by a pressure sensor of the personal device received by the controller node may be used by the controller node to determine the floor on which the individual is located which, in turn, is an input factor to the step of determining, by the controller node for the personal device 200, whether to change connection node for the personal device 200.

In embodiments and in order to improve the determination of whether to change connection node for a personal device 200, the determination of the location and/or whether to change connection node may be improved by involving also signals from a sensor 220 of the personal device 200. The sensor 220 may e.g. be a movement sensor used as a pedometer, so that the signals from the movement sensor 220 indicates how many steps the individual has taken. If the individual is determined to be in a certain location (such as e.g. the bathroom 720), and the amount of steps required for moving to this location from a previously determined location (such as e.g. the bedroom 730) is more than the amount of steps that have been detected by the movement sensor 220, it is likely that the determined location is incorrect. The at least one processing device 310 of a network node (301, 302, 303, 304, 305, 306) may thus be arranged to determine the location of the personal device 200 influencing the decision by the controller node whether to change connection node for the personal device 200 based also on an indication of the movement of the individual in relation to a previously determined location, based on sensor data from the at least one sensor 220.

The processing of the sensor data from the at least one sensor 220 may be done by a trained machine learning algorithm. When many individuals are monitored using many personal devices 200, large amounts of sensor data is collected from the sensors 220 in these personal devices 200. Very efficient evaluation of this sensor data may be done using machine learning, in order to determine a suitable machine learning algorithm.

The updating of the activity state and/or health state for the monitored individual may be incremental, so that only differences in relation to the previously determined activity state and/or health state need to be determined.

The determination of the location for the personal device 200 and/or the by the controller node to change connection node for the personal device 200 may e.g. be done by a trained machine learning algorithm. Machine learning is an efficient way of evaluating complex signals in situations where large amounts of data is collected.

When the determining of whether to change connection node is based on received sensor data indicates a certain location and whether the determined location fulfils a predetermined location condition, the predetermined location condition may be an absolute condition, such that if the determined location does not fulfil the predetermined location condition, the controller node will, or will not, determine to change connection node for the personal device 200. However, there may instead be weighing factors, so that if the received sensor data strongly indicates a specific location, and/or the determined location does not strongly indicate a specific location (that does not fulfil the predetermined location condition), the controller node will, or will not, determine to change connection node for the personal device 200.

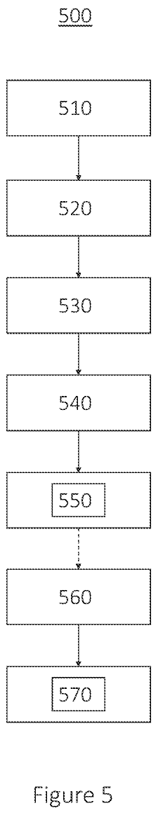

FIG. 5 schematically illustrates embodiments of the technology disclosed including a method (500) for monitoring an individual within predetermined facilities (700) in order to determine whether there is a need for assigning roles to the nodes of a node network comprising at least three network nodes in a monitoring system configured for changing connection node assigned to a personal device worn by the individual, the method (500) comprising:
a. assigning (510) only one node among the at least three network nodes to be the controller node having the sole responsibility for a particular personal device;
b. assigning (520) a connection node for establishing a short-range wireless communication connection between the connection node and the personal device;
c. establishing (530) a short-range wireless communication connection with the personal device;
d. transmitting (540), via the established short-range wireless communication connection, a message to the connection node;
e. receiving (550), by the connection node, the message, wherein the message contains data including at least one of status data, sensor data and event data associated with the personal device;
f. transmitting (560), from the assigned connection node to the sole controller node for the personal device, the received message or a message including data corresponding to, or at least partly based on, the at least one of status data, sensor data and event data associated with said personal device; and
g. transmitting (570), from the controller node to at least one of at least one of the other at least three network nodes and/or a backend system, a message containing data adapted to be used for at least one of controlling and monitoring the personal device, wherein the message is at least partly based on the data corresponding to, or at least partly based on, the at least one of status data, sensor data and event data associated with the personal device.

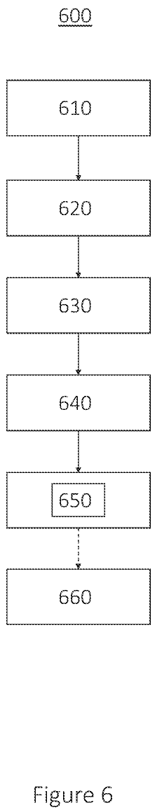

FIG. 6 schematically illustrates embodiments of the technology disclosed including a method (600) for monitoring an individual within predetermined facilities (700) in order to determine whether to assign a new connection node to a personal device (200) in a monitoring system, the method (600) comprising:
a. transmitting (610), by the personal device, short-range wireless broadcast signals;
b. receiving (620), by a plurality of nodes within short-range wireless communication range with the personal device, short-range wireless broadcast signals;
c. measuring (630), by each of the plurality of nodes, signal strengths of the received short-range wireless broadcast signals;
d. transmitting (640), from each of the plurality of nodes and via the IP-based network connecting the at least three nodes, data to the controller node, wherein the data indicates the measured signal strengths values;
e. receiving (650), by the controller node and via the IP-based network, the transmitted data indicating said measured signal strengths values; and
f. determining (660), by the only one controller node, to change connection node assigned to said personal device at least partly based on the received data indicating the measured broadcast signal strengths values, wherein the new connection node for the personal device is a different node from the current connection node.

In aspects, the monitoring system is configured so that the new connection node may be a different node from the current controller node for the personal device but may also be the controller node for the personal device.

In embodiments of the technology disclosed, the monitoring system is configured so that the connection node assigned to a personal device is never the assigned controller node for the personal device.

In embodiments of the technology disclosed, the monitoring system is configured so that the connection node assigned to a personal device is never the currently assigned controller node for the personal device, but the controller node and connection node assigned to a personal device may be dynamically changed so that a node that previously was connection node for a personal device may be the controller node for the personal device, and vice versa.

In certain aspects and embodiments of the technology disclosed, the monitoring system is configured so that the connection node assigned to a personal device may, from time to time, also be the currently assigned controller node for the personal device. According to these embodiments, the connection node assigned to the respective personal devices is dynamically changed so that a network node that previously was both controller node and connection node for the same personal device may later on be only controller node for the personal device and not the controller node for the personal device.

In certain aspects and embodiments of the technology disclosed, the monitoring system is configured so that the connection node assigned to a personal device may, from time to time, also be the currently assigned controller node for the personal device. According to these embodiments, both the connection node and controller node assigned to the respective personal devices is dynamically changed so that a network node that previously was both controller node and connection node for the same personal device may later on be neither connection node nor controller node for the personal device.

In aspects, the technology disclosed relates to a method and system for monitoring an individual within predetermined facilities (700):
receiving, by the sole connection node assigned to the personal device, a message comprising sensor data obtained by at least one sensor (220) arranged in the personal device (200);
forwarding, by the connection node and to the controller node assigned to the personal device, a message comprising the sensor data or data reflecting the sensor data;
determining, by the controller node whether the received sensor data indicates certain event data, a specific health state and/or a specific alert state among a plurality of predetermined alert states, where the alert states may comprising at least a FALL alert state, to be used if a probable fall has been detected for the monitored individual, and an OUT OF BED alert state, to be used if it has been detected that the monitored individual is probably getting out of bed;receiving, by a plurality of nodes within short-range wireless communication range with the personal device, short-range wireless broadcast signals from the personal device;
measuring, by each of the plurality of nodes, signal strengths values of the received short-range wireless broadcast signals;
forwarding, by each of the plurality of nodes and to the controller node assigned to the personal device, a message containing the signal strengths values of the received short-range wireless broadcast signals;
determining, by the controller node, the location of the personal device (200) within the facilities (700) based at least on the received signal strengths values of the short-range wireless broadcast signals together with information retrieved from a storage means (320) regarding the layout of the facilities (700) within which the monitoring of the individual takes place; and
setting an alert and/or change a health state based at least on whether the received sensor data indicates a specific event, health state and/or an alert state and whether the determined location fulfils a predetermined location condition, indicating whether the individual is located in a certain room, such as e.g. the bedroom (630).

According to embodiments of the various methods mentioned above, the determining of the location of the personal device (200) is based also on an indication of the movement of the individual in relation to a previously determined location, based on sensor data from the at least one sensor (220), e.g. a motion sensor and/or a pressure sensor.

According to embodiments of the technology disclosed and mentioned above, the method further comprising continuously determining and updating an activity state and/or a heath state for the monitored individual.

According to embodiments of the technology disclosed and mentioned above, the method further the change of health state and/or setting of the alert is based also on at least one previously determined health state or activity state. In the example of setting an alert state, the setting of an OUT OF BED alert state may also depend on whether the previous activity state was a LYING IN BED alert state, or so that the setting of a FALL alert state also depends on that the previous activity state was not any type of LYING activity state.

According to embodiments of the technology disclosed and mentioned above, the changed health state and/or setting of the alert is based also on the time period for which the individual has had a specific activity state, such as e.g. for how long the individual has been in the activity state STANDING.

According to embodiments of the technology disclosed and mentioned above, determining of the changed health state and/or activity state is done by a trained machine learning algorithm.

According to embodiments of the technology disclosed and mentioned above, the method further comprises sending an alarm signal if a predetermined alert or health state is set.

In embodiments, the communication between the personal device (200) and the at least one network node (300) takes place using a personal device communication interface (230) and a network node communication interface (330).

In aspects, the technology disclosed relates to a system (100) for monitoring an individual within predetermined facilities (700), the system (100) comprising:
a personal device (200) to be worn by the monitored individual, the personal device (200) comprising at least one sensor or input means (220);
at least one network node (300), with which the personal device (200) is arranged to communicate via an established short-range wireless connection;
a storage means (320); and
at least one processing device (210, 310, 160), arranged to:
   receive sensor data, event data and/or input data from the at least one sensor and/or input means (220) in the personal device (200);
   determine whether the received sensor data, event data and/or input data indicates a specific health state and/or an alert state among a plurality of health states and/or predetermined alert states; and;
   set a health state and/or an alert based at least on whether the received sensor data, event data and/or input data indicates a health state and/or an alert state.

In embodiments, the system (100) and controller node for the personal device (200) are configured to set a health state and/or an alert based at least on whether a determined location fulfils a predetermined location condition, indicating whether the individual is located in a certain room, such as e.g. the bedroom (630).

In embodiments, the system (100) and controller node for the personal device (200) are configured to determine the distance between the personal device (200) and the at least one network node (300), e.g. based on signal strength values measured by at least one of the network nodes.

In embodiments, the system (100) and controller node for the personal device (200) are configured to determine the location of the personal device (200) within the facilities (700) based at least on the determined distance together with information retrieved from the storage means (320) regarding the layout of the facilities (700) within which the monitoring of the individual takes place.

In embodiments, the system (100) and the at least one processing device (210, 310, 160) are arranged to continuously determine and update a health state and/or an activity state for the monitored individual based on received sensor data is indicating a specific event, health state and/or an alert state.

In embodiments, the personal device (200) comprises at least one motion sensor (220). In certain embodiment the at least one motion sensor (220) is an accelerometer.

In embodiments, the personal device (200) comprises at least one medical sensor (220), or health sensor, where the medical sensor (220) is adapted to obtain health sensor data to be used for determining the current health status or health state of the individual wearing the personal device.

In embodiments, the personal device (200) is a wristworn device, such as e.g. a wristband, clip or necklace.

An accelerometer, is a device that measures proper acceleration. Proper acceleration, being the acceleration (or rate of change of velocity) of a body in its own instantaneous rest frame, is not the same as coordinate acceleration, being the acceleration in a fixed coordinate system.

The input from the movement sensor based on sensor data indicating an acceleration beyond a certain threshold value and/or a certain changed movement pattern for the personal device may trigger the short-range wireless transmission of event data from the personal device.

1. A method in a monitoring system including a network comprising at least three network nodes and a plurality of personal devices with short-range wireless communication capabilities, said method comprising:
a assigning only one node among the at least three network nodes to be the controller node having the sole responsibility for a particular personal device;
b assigning, by said controller node, a connection node for establishing a short-range wireless communication connection between said connection node and said personal device;
c establishing, by said assigned connection node, a short-range wireless communication connection with said personal device;
d transmitting, from said personal device and via said established short-range wireless communication connection, a message to said connection node;
e receiving, by said connection node, said message, wherein said message contains data including at least one of status data, sensor data and event data associated with said personal device;
f transmitting, from the assigned connection node to the sole controller node for said personal device, said received message or a message including data corresponding to, or at least partly based on, said at least one of status data, sensor data and event data associated with said personal device; and
g transmitting, from said controller node to at least one of at least one of the other at least three network nodes and a backend system, a message containing data adapted to be used for at least one of controlling and monitoring the personal device, wherein said message is at least partly based on said data corresponding to, or at least partly based on, said at least one of status data, sensor data and event data associated with said personal device.

2. The method of claim 1, wherein said short-range wireless communication connection is the only currently established communication connection between the network nodes and said personal device.

3. The method of claim 1, wherein each of the plurality of personal devices is assigned only one of the at least three network nodes to be the controller node for the respective personal device.

4. The method of claim 1 wherein each of the plurality of personal devices is assigned, by their respective controller node, only one of the at least three network nodes to be the connection node for the personal device.

5. The method of claim 1, wherein each of the at least three network nodes has no more than one established short-range wireless connection with a personal device at a time.

6. The method of claim 1, further comprising distributing or sharing data between the at least three network nodes via an IP-based network not using the same short-range wireless communication protocols used by the personal devices for communicating with the node network, wherein said distributing or sharing of data between the nodes is preceding the assignment of the controller node, and wherein the data distributed or shared between the at least three network nodes is used for determining which of the at least three nodes is going to be the controller node for the personal device.

7. The method of claim 6, wherein said distributing or sharing of data between the nodes is preceding the assignment of the controller node, and wherein the data distributed or shared between the at least three network nodes is used for determining which of the at least three nodes is going to be the controller node for the personal device.

8. The method of claim 6, further comprising determining which of the at least three nodes is going to be the controller node for the personal device by using a common decision model which is known to each of the at least three nodes.

9. The method of claim 8, wherein said common decision model use input values or parameters which are continuously and dynamically changed at least partly based on data distributed and shared between the network nodes, thereby allowing for the determining of which of the nodes among the at least three nodes is going to be the controller node for the personal device to be dynamically changed at least partly on data distributed and shared between the network nodes.

10. The method of claim 1, further comprising:
a transmitting, from said personal device and via said established short-range wireless communication connection, a message to said assigned connection node;
b receiving, by said connection node, said message, wherein said message contains data including at least one of status data, sensor data and event data associated with said personal device;
c transmitting, from the assigned connection node to the sole controller node for said personal device, said received message or a message including data corresponding to, or at least partly based on, said at least one of status data, sensor data and event data associated with said personal device; and
d transmitting, from said controller node to at least one of at least one of the other at least three network nodes and a backend system, a message containing data adapted to be used for at least one of controlling and monitoring the personal device, wherein said message is at least partly based on said data corresponding to, or at least partly based on, said at least one of status data, sensor data and event data associated with said personal device.

11. The method of claim 10, further comprising the following step prior to transmitting said message containing data adapted to be used for at least one of controlling and monitoring the personal device:
a determining, by said controller node, whether to make any decisions or take any actions associated with and/or on behalf of said personal device at least partly in response to said received message and at least partly based on said received at least one of status data, sensor data and event data associated with said personal device.

12. The method of claim 1, further comprising:
a transmitting, from said personal device, short-range wireless broadcast signals.

13. The method of claim 12, wherein said short-range wireless broadcast signals are transmitted while said personal device has an established short-range wireless communication connection with the currently assigned connection node.

14. The method of claim 12, further comprising forwarding, by at least a plurality nodes within short-range wireless communication range with the personal device, at least a portion of the contents of said short-range wireless broadcast signals to the other network nodes, wherein the at least a portion of the contents of said broadcast signals is used by the network of nodes as input values to the for determining which of the at least three nodes is going to be the controller node for the personal device.

15. The method of claim 12, further comprising forwarding, by at least a plurality nodes within short-range wireless communication range with the personal device, at least a portion of the contents of said short-range wireless broadcast signals to the controller node, wherein the contents of said broadcast signals is adapted to be used by the controller node for determining which of the at least three nodes is to be assigned connection node for the personal device.

16. The method of claim 12, comprising:
a receiving, by a plurality of nodes within short-range wireless communication range with the personal device, said short-range wireless broadcast signals;
b measuring, by each of said plurality of nodes, signal strengths of said received short-range wireless broadcast signals;
c transmitting, from each of said plurality of nodes and via the IP-based network connecting the at least three nodes, data to said controller node, wherein said data indicates said measured signal strengths values;
d receiving, by said controller node and via said IP-based network, said transmitted data indicating said measured signal strengths values; and
e determining, by said only one controller node, to change connection node assigned to said personal device at least partly based on said received data indicating said measured broadcast signal strengths values, wherein said new connection node for said personal device is a different node from the current connection node and the current controller node for said personal device.

17. The method of claim 16, further comprising:
a determining, by said only one controller node, to change the only one connection node for said personal device at least partly based on said received data indicating said measured broadcast signal strengths values, wherein said new only one connection node is assigned for establishing a new short-range wireless communication connection between said new only one connection node and said personal device; and
b establishing, by said new only one connection node for said personal device, a new short-range wireless communication connection, wherein said new short-range wireless connection is the only currently established short-range wireless connection between said node network and said personal device.

18. A monitoring system including a network comprising at least three network nodes configured to transmit data to the other nodes via the network and a plurality of personal devices with short-range wireless communication capabilities for communicating with the at least three network nodes, said plurality of personal devices are each assigned only one controller node among the at least three network nodes, wherein the respective controller node is responsible for assigning a connection node among the at least three network nodes to establish a short-range wireless communication connection with a personal device the controller node is responsible for.

19. The monitoring system according to claim 18, wherein said monitoring system is configured so that each of said plurality of personal devices is assigned only one controller node which has the sole responsibility for the personal device.

20. The monitoring system according to claim 19, wherein said monitoring system is configured so that each or at least one of the at least three network nodes may be the assigned sole controller node for a plurality of personal devices.

21. The monitoring system according to claim 18, wherein said monitoring system including the at least three network nodes is configured to use a common decision model which is known to each of the at least three nodes for determining which of the at least three nodes is going to be the controller node assigned to the respective personal device.

22. The monitoring system according to claim 21, wherein said common decision model use input values or parameters which are continuously and dynamically changed at least partly based on data distributed and shared between the network nodes, thereby allowing for the determining of which of the nodes among the at least three nodes is going to be the controller node for the personal device to be dynamically changed at least partly on data distributed and shared between the network nodes.

23. The monitoring system according to claim 18, wherein said monitoring system is configured so that each assigned network node to be connection node for a personal device only has one established short-range wireless communication connection with the node network at a time.

24. The monitoring system according to claim 18, wherein said plurality of personal devices are each configured to be transmitting short-range wireless broadcast signals while having an established short-range wireless communication connection with their respective connection node.

25. The monitoring system according to claim 18, wherein the only one controller node assigned to the respective personal device is configured to assign and dynamically change which of the at least three network nodes is currently the only one connection node for the personal device at least partly based on at least one of contents and received signal strengths of at least one short-range wireless broadcast signal transmitted from the personal device and which is received by a plurality of other nodes than the controller node.

26. The monitoring system according to claim 18, wherein the only one controller node assigned to the respective personal device is configured to receive data from a plurality of other nodes than the controller node via an IP-based network connecting the at least three network nodes, and wherein said data received via said IP-based network include data indicating the measured signal strength values of short-range wireless broadcast signals transmitted by the respective personal device and which are received and measured by the plurality of other nodes.

27. The monitoring system according to claim 26, wherein the only one controller node assigned to the respective personal device is further configured to change the only one connection node for the personal devices the only one controller node is responsible for at least partly based on said data indicating the measured signal strength values of short-range wireless broadcast signals transmitted by the respective personal device and which are received and measured by the plurality of other nodes.

28. The monitoring system according to claim 18, the network comprising at least three network nodes configured to transmit data to the other nodes via the network is an IP-based network and the short-range wireless communication capabilities of the plurality of personal devices for communicating with the at least three network nodes are Bluetooth communication capabilities.

29. The monitoring system according to claim 18, wherein said plurality of personal devices are at least one of wristbands, necklaces and clips.
